(19) United States
(12) Patent Application Publication
(10) Pub. No.: US 2021/0095029 A1
(43) Pub. Date: Apr. 1, 2021

(54) COMPOSITIONS AND METHODS FOR TREATING CANCER

(71) Applicant: University of Southern California, Los Angeles, CA (US)

(72) Inventors: Pin WANG, Los Angeles, CA (US); Natnaree SIRIWON, Los Angeles, CA (US); Si LI, Los Angeles, CA (US)

(73) Assignee: University of Southern California, Los Angeles, CA (US)

(21) Appl. No.: 16/603,792

(22) PCT Filed: Apr. 19, 2018
(86) PCT No.: PCT/US2018/028427

§ 371 Date: Oct. 8, 2019

Related U.S. Application Data
(60) Provisional application No. 62/487,358, filed on Apr. 19, 2017.

(51) Int. Cl. C07K 16/28; A61K 35/17; A61P 35/00; A61K 45/06; A61K 39/395; A61K 38/17; C07K 14/705; C07K 14/725

(52) U.S. Cl. CPC C07K 16/2818; A61K 35/17; A61P 35/00; A61K 45/06; A61K 39/3955; A61K 38/1774; A61K 2039/507; C07K 14/70517; C07K 14/70521; C07K 14/7051; C07K 2317/622; C07K 2317/76; C07K 16/2803

(57) ABSTRACT

Described herein are compositions a genetically modified comprising nucleic acids encoding a chimeric antigen receptor (CAR) and a checkpoint inhibitor and methods for using the compositions to treat cancer.

### CROSS-REFERENCE TO RELATED APPLICATIONS

This application includes a claim of priority under 35 U.S.C. § 119(e) to U.S. provisional patent application No. 62/487,358, filed Apr. 19, 2017, the entirety of which is hereby incorporated by reference.

### STATEMENT REGARDING FEDERALLY SPONSORED RESEARCH OR DEVELOPMENT

This invention was made with government support under Grant Nos. AI068978 and EB017206 awarded by National Institutes of Health. The government has certain rights in the invention.

### TECHNICAL FIELD

Described herein are compositions which include T cells comprising chimeric antigen receptors (CARs) and checkpoint inhibitors (CPIs) and methods for using the compositions to treat cancer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Adoptive cell transfer (ACT), as a modality of immunotherapy for cancer, has demonstrated remarkable success in treating hematologic malignancies and malignant melanoma. An especially effective form of ACT, which uses gene-modified T cells expressing a chimeric antigen receptor (CAR) to specifically target tumor-associated-antigen (TAA), such as CD19 and GD2, has displayed encouraging results in clinical trials for treating such diseases as B cell malignancies and neuroblastoma.

Unlike naturally occurring T cell receptors (TCRs), CARs are artificial receptor consisting of an extracellular antigen recognition domain fused with intracellular T cell signaling and costimulatory domains. CARS can directly and selectively recognize cell surface TAAs in a major histocompatibility class (MHC)-independent manner. Despite the documented success of CAR T cell therapy in patients with hematologic malignancies, only modest responses have been observed in solid tumors. This can be attributed, in part, to the establishment of an immunosuppressive microenvironment in solid tumors. Such milieu involves the upregulation of a number of intrinsic inhibitory pathways mediated by increased expression of inhibitory receptors (IRs) in T cells reacting with their cognate ligands within the tumor.

So far, several IRs have been characterized in T cells, such as CTLA-4, T cell Ig mucin-3 (TIM-3), lymphocyte-activation gene 3 (LAG-3), and programmed death-1 (PD-1). These molecules are upregulated following sustained activation of T cells in chronic disease and cancer, and they promote T cell dysfunction and exhaustion, thus resulting in escape of tumor from immune surveillance. Unlike other IRs, PD-1 is upregulated shortly after T cell activation, which in turn, inhibits T cell effector function via interacting with its two ligands, PD-L1 or PD-L2. PD-L1 is constitutively expressed on T cells, B cells, macrophages, and dendritic cells (DCs). PD-L1 is also shown to be abundantly expressed in a wide variety of solid tumors. In contrast, the expression of PD-L1 in normal tissues is undetectable. As a consequence of its critical role in immunosuppression, PD-1 has been the focus of recent research, aiming to neutralize its negative effect on T cells and enhance antitumor responses. Clinical studies have demonstrated that PD-1 blockade significantly enhanced tumor regression in colon, renal and lung cancers and melanoma.

Therefore, it is an objective of the present invention to provide a composition that modulates tumor-induced hypofunction of CAR T cells, and may reverse or inhibit the inhibitory receptors.

It is another objective of the present invention to provide a process of making and using a composition that modulates or avoids tumor-induced hypofunction of CAR T cells.

### SUMMARY

The following embodiments and aspects thereof are described and illustrated in conjunction with systems, compositions and methods which are meant to be exemplary and illustrative, not limiting in scope.

A cell is provided containing a nucleic acid encoding both a chimeric antigen receptor (CAR) and a checkpoint inhibitor (CPI) or containing a nucleic acid encoding a CAR and a nucleic acid encoding a CPI. In various embodiments, CAR-T cells secreting checkpoint inhibitors are provided.

In various embodiments, CAR-T cells secreting checkpoint inhibitors (CPIs) targeting PD-1 (denoted as CAR.αPD1-T cells) are provided and shown of their efficacy in a human lung carcinoma xenograft mouse model. Despite favorable responses of chimeric antigen receptor (CAR)-engineered T cell therapy in patients with hematologic malignancies, the outcome has been far from satisfactory in the treatment of solid tumors, partially owing to the development of an immunosuppressive tumor microenvironment. In some aspects, in order to overcome the inhibitory effect of PD-1 signaling in CAR T cells, genetically engineered CAR T cells with the capacity to continuously produce a single-chain variable fragment (scFv) form of anti-PD-1 antibody are used. In tumor models, anti-PD-1 scFv expression and secretion interrupt the engagement of PD-1 with its ligand, PD-L1, and prevent CAR T cells from being inhibited and exhausted. In a CD19 tumor model, the secretion of anti-PD-1 scFv by CAR T cells significantly improves the capacity of CAR T cells in eradicating an established solid tumor.

Typically, CAR.αPD1-T cells demonstrate the effector function and expansion capacity, as measured by the production of IFN-γ and T cell proliferation following antigen-specific stimulation. The antitumor efficacy of CAR.αPD1-T cells is superior than CAR-T cells alone or CAR-T cells combined with anti-PD-1 antibody using a xenograft mouse model. The enhanced tumor eradication of CAR.αPD1-T cells is further supported by the expansion and functional capacity of tumor-infiltrating lymphocytes.

In various embodiments, CAR.αPD1-T cells secrete human anti-PD-1 CPIs which efficiently bind to PD-1 and reverse the inhibitory effect of PD-1/PD-L1 interaction on T cell function. PD-1 blockade by continuously secreted anti-PD-1 prevents T cell exhaustion and significantly enhances T cell expansion and effector function both in vitro and in vivo. In the xenograft mouse model, the secretion of anti-PD-1 enhances the antitumor activity of CAR-T cells and prolongs overall survival. With constitutive anti-PD-1 secretion, CAR.αPD1-T cells are less exhausted, more functional and expandable, and more efficient at tumor eradication than parental CAR-T cells.

A process is provided where a cell containing nucleic acids encoding a CAR and a CPI is administered to a subject in need thereof to enhance antitumor immunity and/or to treat cancer (especially reducing solid tumors).

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1E depict construction and characterization of CAR19 and CAR19.αPD1. FIG. 1A shows a schematic representation of parental anti-CD19 CAR (CAR19) and anti-PD-1-secreting anti-CD19 CAR (CAR19.αPD1) constructs. FIG. 1B shows the expression of both CARs in human T cells. The two groups of CAR T cells were stained with biotinylated protein L followed by FITC-conjugated streptavidin to detect CAR expression on the cell surface. A viable CD3⁺ lymphocyte gating strategy was used. NT indicates nontransduced T cells, which were used as a control. FIGS. 1C and 1D show the expression of secreted anti-PD-1 antibody in the supernatant from either CAR19 or CAR19.αPD1 T cell culture as analyzed by Western blot (1C) and ELISA (1D). FIG. 1E shows the percentage of CD8⁺ T cells expressing IFN-γ over total CD8⁺ T cells with the indicated treatment (n=4, mean±SEM; **P<0.01).

FIGS. 2A-2D depict anti-PD-1 expression enhanced the antigen-specific immune responses of CAR T cells. FIG. 2A shows both CAR19 and CAR19.αPD1 T cells were cocultured with H292-CD19 cells for different durations. IFN-γ production was measured by ELISA (n=5, mean±SEM; ns, not significant, P>0.05; *P<0.05). FIG. 2B shows cytotoxicity of both CARs against target cells. The two groups of CAR T cells were cocultured for 6 hours with H292-CD19 cells at 1:1, 5:1, 10:1, and 20:1 effector-to-target ratios, and cytotoxicity against H292-CD19 was measured. Nontransduced (NT) T cells were used as a control. FIG. 2C shows proliferation of both CARs after antigen-specific stimulation. The two groups of CAR T cells were pre-stained with CFSE. The stained T cells were then cocultured for 96 hours with H292-CD19 cells at 1:1 effector-to-target ratio and the intensity of CFSE was measured. Nontransduced (NT) cells were used as a control. FIG. 2D shows the summarized statistics in bar graphs of proliferation rate for nontransduced (NT) T cells, CAR19 T cells, and CAR19.αPD1 T cells corresponding to FIG. 2C (n=4, mean±SEM; *P<0.05).

FIGS. 3A-3F depict secreting anti-PD-1 scFv protected CAR T cells from being exhausted. Both CAR19 and CAR19.αPD1 T cells were cocultured with H292-CD19 cells for 24 hours. FIG. 3A shows PD-1 expression as measured by flow cytometry. CD8⁺ T cells were shown in each panel. PD-1-expressing CD8 T cells were gated, and their percentage over total CD8⁺ T cells was shown in each scatterplot. FIG. 3B shows the summarized statistics of triplicates in bar graphs (n=3, mean±SEM; **P<0.01; ***P<0.001). FIG. 3C shows LAG-3 expression measured by flow cytometry. The percentage of LAG-3-expressing CD8 T cells over total CD8⁺ T cells was shown in bar graphs (n=3, mean±SEM; ns, not significant, P>0.05; **P<0.01). FIG. 3D shows TIM-3 expression as measured by flow cytometry. The percentage of TIM-3-expressing CD8 T cells over total CD8⁺ T cells was shown in bar graphs (n=3, mean±SEM; ns, not significant, P>0.05). FIGS. 3E and 3F depict that both CAR19 and CAR19.αPD1 T cells were cocultured with either H292-CD19 or SKOV3-CD19 cells for 24 hours. PD-L1 expression was measured by flow cytometry. The percentages of PD-L1-expressing CD8 T cells over total CD8⁺ T cells (FIG. 3E) and PD-L1-expressing CD4 T cells over total CD4⁺ T cells (FIG. 3F) were shown in bar graphs (n=3, mean±SEM; *P<0.05; **P<0.01; ***P<0.001).

FIGS. 4A-4D depict adoptive transfer of CAR T cells secreting anti-PD-1 scFv enhanced the growth inhibition of established tumor. FIG. 4A shows schematic representation of the experimental procedure for tumor challenge, T cell adoptive transfer and antibody treatment. NSG mice were s.c. challenged with 3×10⁶ of H292-CD19 tumor cells. At day 20, when the tumors grew to ˜100 mm³, 1×10⁶ of CAR19 or CAR19.αPD1 T cells were adoptively transferred through i.v. injection. One day post-T cell infusion, anti-PD-L1 antibody treatment was initiated, and the treatment was continued on the indicated dates. Tumor volume was measured every other day. FIG. 4B shows tumor growth curve for mice treated with nontransduced (NT), NT plus anti-PD-1 injection, CAR19, CAR19 plus anti-PD-1 injection, or CAR19.αPD1. Data were presented as mean tumor volume±standard error of the mean (SEM) at indicated time points (n=8; *P<0.05; ***P<0.001). FIG. 4C shows waterfall plot analysis of tumor reduction on day 17 post-therapy for various treatment groups. FIG. 4D shows survival of H292-CD19 tumor-bearing NSG mice after indicated treatment. Overall survival curves were plotted using the Kaplan-Meier method and compared using the log-rank (Mantel-Cox) test (n=6; ns, not significant, P>0.05; *P<0.05; **P<0.01).

FIGS. 5A-5C depict CAR T cells secreting anti-PD-1 expanded more efficiently than parental CAR T cells in vivo. The percentage of human CD45⁺ T cells in the tumor, blood, spleen and bone marrow of H292-CD19 tumor-bearing mice that were adoptively transferred with nontransduced (NT), CAR19, or CAR19.αPD1 T cells was investigated by flow cytometry at day 2 (5A) or day 10 (5B) post-therapy (n=3, mean±SEM; *P<0.05; ***P<0.001). FIG. 5C shows a representative FACS scatter plot of the percentage of human CD45⁺ T cells in the tumor, blood, spleen and bone marrow of different groups.

FIGS. 6A-6G depict CAR T cells secreting anti-PD-1 were more functional than parental CAR T cells at local tumor site. FIG. 6A shows a schematic representation of the experimental procedure for tumor challenge, T cell adoptive transfer and antibody treatment. NSG mice were s.c. challenged with 3×10⁶ of H292-CD19 tumor cells. At day 20, 3×10⁶ of CAR19 or CAR19.αPD1 T cells were adoptively transferred through i.v. injection. One day post-T cell adoptive transfer, anti-PD-1 antibody treatment was initiated, and the treatment was continued on the indicated dates. The mice were then euthanized on day 8 for analysis. FIG. 6B shows the percentage of human CD45⁺ T cells in the tumor, blood, spleen and bone marrow of H292-CD19 tumor-bearing mice that were adoptively transferred with CAR19 or CAR19.αPD1 T cells, or treated with CAR19 T cells along with injection of anti-PD-1 antibody, as characterized by flow cytometry. FIG. 6C shows the ratio of CD8⁺ versus CD4⁺ TILs in the tumor (n=3, mean±SEM; ns, not significant, P>0.05; *P<0.05; ***P<0.001). FIG. 6D shows the percentage of PD-1-expressing CD8 TILs over total CD8⁺ TILs (n=3, mean±SEM; *P<0.05). TILs were harvested and stimulated ex vivo for 6 hours by either anti-CD3/anti-CD28 antibodies (6E) or target cells H292-CD19 (6F). The percentage of CAR T cells in the tumor expressing intracellular IFN-γ was investigated by flow cytometry (n=3, mean±SEM; *P<0.05; **P<0.01). FIG. 6G shows the secreted anti-PD-1 scFvs and injected anti-PD-1 antibodies in the sera as evaluated using ELISA (n=3, mean±SEM; **P<001; ***P<0.001).

FIG. 7A depicts the production of anti-PD-1 scFv from CAR19.αPD1 T cells (1×10⁶) after 24-hour culture with or without Brefeldin A. FIG. 7B depicts the expression of anti-PD-1 scFv during the course of CAR19.αPD1 cell expansion. The concentration of secreted scFv was measured at four different time points post T cell transduction, including days 4, 7, 10 and 12. The cell density was maintained around 2-4×10⁶ per ml during T cell expansion. FIG. 7C depicts human T cells were activated with anti-CD3/CD28 beads for 48 hours and then cultured in T cell culture medium supplemented with 10 ng/ml of human IL-2 for two weeks. The activated T cells were then stained with either isotype control antibody or anti-PD-1 antibody. FIG. 7D depicts the activated human T cells were incubate with 1 ml of CAR19.αPD1 cell culture supernatant for 30 min. The cells was washed once with PBS and then stained with anti-HA antibody.

FIG. 8 depicts the expression of PD-L1 on H292-CD19 and SKOV3-CD19 as determined by flow cytometry.

FIG. 9A depicts both CAR19 and CAR19.αPD1 T cells were cocultured with SKOV3-CD19 cells for different durations. IFN-γ production was measured by ELISA (n=5, mean±SEM; ns, not significant, P>0.05; *P<0.05). FIG. 9B depicts CAR19 cells with or without anti-PD-1 (0.6 μg/ml), and CAR19.αPD1 T cells were cocultured with H292-CD19 cells for 24 or 72 hours. IFN-γ production was measured by ELISA (n=4, mean±SEM; ns, not significant, P>0.05; ***P<0.001).

FIG. 10 depicts the population doublings of nontransduced (NT), CAR19 and CAR19.αPD1 T cells upon antigen-specific stimulation for 3 days (n=3, mean±SEM; **P<0.01).

FIG. 11A depicts the blocking activity of anti-PD-1 say on the binding of PD-1 detection antibody. Human T cells were activated with anti-CD3/CD28 beads for 48 hours and then cultured in TCM supplemented with 10 ng/ml of human IL-2 for two weeks. The activated T cells were then incubated with 1 ml of CAR19.αPD1 cell culture supernatant or control medium for 30 min. The T cells were washed once with PBS and then stained with anti-PD-1 antibody. FIG. 11B depicts the relative transcriptional expression of PD-1 on CAR19 and CAR19.αPD1 T cells upon antigen-specific stimulation for 24 hours (n=3, mean±SEM; ***P<0.001).

FIGS. 12A and 12B depict the representative gating schemes and plots for CD8⁺PD-L1⁺ cells (12A) and CD8⁺LAG-3⁺ and CD8⁺TIM-3⁺ T cells (12B) after antigen-specific stimulation for 24 hours.

FIGS. 13A-13E depict that both CAR19 and CAR19.αPD1 T cells were cocultured with H292-CD19 cells for 24 hours. The expression of PD-1 (13A), LAG-3 (13B) and TIM-3 (13C) was measured by flow cytometry. The percentage of PD-1-, LAG-3- or TIM-3-expressing CD4 T cells over total CD4⁺ T cells was shown in bar graphs (n=3, mean±SEM; ns, not significant, P>0.05; **P<0.01). The expression of PD-1 (13D) and LAG-3 (13E) in both CAR19 and CAR19.αPD1 T cells during the course of T cell expansion (post T activation and transduction).

FIG. 14A depicts the ratio of CD8⁺ versus CD4⁺ T cells before they were adoptively transferred into the mice. FIG. 14B depicts the ratio of CD8⁺ versus CD4⁺ T cells from the mice treated with CAR19.αPD1 T cells (n=3, mean±SEM; **P<0.01). FIG. 14C depicts the expression of IFN-γ in the sera was measured by ELISA.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul. 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

### Definitions

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

As used herein, the term “about” refers to a measurable value such as an amount, a time duration, and the like, and encompasses variations of ±20%, ±10%, ±5%, ±1%, ±0.5% or ±0.1% from the specified value.

“Chimeric antigen receptor” or “CAR” or “CARs” as used herein refers to engineered receptors, which graft an antigen specificity onto cells (for example T cells such as naïve T cells, central memory T cells, effector memory T cells or combination thereof). CARs are also known as artificial T-cell receptors, chimeric T-cell receptors or chimeric immunoreceptors. In various embodiments, CARs are recombinant polypeptides comprising an antigen-specific domain (ASD), a hinge region (HR), a transmembrane domain (TMD), co-stimulatory domain (CSD) and an intracellular signaling domain (ISD).

“Antigen-specific domain” (ASD) refers to the portion of the CAR that specifically binds the antigen on the target cell. In some embodiments, the ASD of the CARs comprises an antibody or a functional equivalent thereof or a fragment thereof or a derivative thereof. The targeting regions may comprise full length heavy chain, Fab fragments, single chain Fv (scFv) fragments, divalent single chain antibodies or diabodies, each of which are specific to the target antigen. In some embodiments, almost any molecule that binds a given antigen with high affinity can be used as an ASD, as will be appreciated by those of skill in the art. In some embodiments, the ASD comprises T cell receptors (TCRs) or portions thereof.

“Hinge region” (HR) as used herein refers to the hydrophilic region which is between the ASD and the TMD. The hinge regions include but are not limited to Fc fragments of antibodies or fragments or derivatives thereof, hinge regions of antibodies or fragments or derivatives thereof, CH2 regions of antibodies, CH3 regions of antibodies, artificial spacer sequences or combinations thereof. Examples of hinge regions include but are not limited to CD8a hinge, and artificial spacers made of polypeptides which may be as small as, for example, Gly3 or CH1 and CH3 domains of IgGs (such as human IgG4). In some embodiments, the hinge region is any one or more of (i) a hinge, CH2 and CH3 regions of IgG4, (ii) a hinge region of IgG4, (iii) a hinge and CH2 of IgG4, (iv) a hinge region of CD8a, (v) a hinge, CH2 and CH3 regions of IgG1, (vi) a hinge region of IgG1 or (vi) a hinge and CH2 region of IgG1. Other hinge regions will be apparent to those of skill in the art and may be used in connection with alternate embodiments of the invention.

“Transmembrane domain” (TMD) as used herein refers to the region of the CAR which crosses the plasma membrane. The transmembrane domain of the CAR of the invention is the transmembrane region of a transmembrane protein (for example Type I transmembrane proteins), an artificial hydrophobic sequence or a combination thereof. Other transmembrane domains will be apparent to those of skill in the art and may be used in connection with alternate embodiments of the invention. In some embodiments, the TMD of the CAR comprises a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

“Co-stimulatory domain” (CSD) as used herein refers to the portion of the CAR which enhances the proliferation, survival and/or development of memory cells. The CARs of the invention may comprise one or more co-stimulatory domains. Each co-stimulatory domain comprises the costimulatory domain of any one or more of, for example, members of the TNFR superfamily, CD28, CD137 (4-1BB), CD134 (OX40), Dap10, CD27, CD2, CD5, ICAM-1, LFA-1(CD11a/CD18), Lck, TNFR-I, TNFR-II, Fas, CD30, CD40 or combinations thereof. Other co-stimulatory domains (e.g., from other proteins) will be apparent to those of skill in the art and may be used in connection with alternate embodiments of the invention.

“Intracellular signaling domain” (ISD) or “cytoplasmic domain” as used herein refers to the portion of the CAR which transduces the effector function signal and directs the cell to perform its specialized function. Examples of domains that transduce the effector function signal include but are not limited to the z chain of the T-cell receptor complex or any of its homologs (e.g., h chain, FceR1g and b chains, MB1 (Iga) chain, B29 (Igb) chain, etc.), human CD3 zeta chain, CD3 polypeptides (D, d and e), syk family tyrosine kinases (Syk, ZAP 70, etc.), src family tyrosine kinases (Lck, Fyn, Lyn, etc.) and other molecules involved in T-cell transduction, such as CD2, CD5 and CD28. Other intracellular signaling domains will be apparent to those of skill in the art and may be used in connection with alternate embodiments of the invention.

“Linker” (L) or “linker domain” or “linker region” as used herein refer to an oligo- or polypeptide region from about 1 to 100 amino acids in length, which links together any of the domains/regions of the CAR of the invention. Linkers may be composed of flexible residues like glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In other embodiments, the linker sequences may comprise Asp-Val/Ile-Glu-X-Asn-Pro-Gly^{(2A)}-Pro^{(2B)} (SEQ ID NO: 1) motif, which results in cleavage between the 2A glycine and the 2B proline. Other linkers will be apparent to those of skill in the art and may be used in connection with alternate embodiments of the invention.

“Autologous” cells as used herein refers to cells derived from the same individual as to whom the cells are later to be re-administered into.

“Genetically modified cells”, “redirected cells”, “genetically engineered cells” or “modified cells” as used herein refer to cells that express the CARs and checkpoint inhibitors. In some embodiments, the genetically modified cells comprise vectors that encode a CAR and vectors that encode one or more checkpoint inhibitors, wherein the two vectors are different. In some embodiments, the genetically modified cells comprise a vector that encodes a CAR and one or more checkpoint inhibitors. In some embodiments, the genetically modified cells comprise a first vector that encodes a CAR and a second vector that encodes the checkpoint inhibitor. In one embodiment, the genetically modified cell is a T-lymphocyte cell (T-cell). In one embodiment, the genetically modified cell is a Natural Killer (NK) cells.

“Immune cell” as used herein refers to the cells of the mammalian immune system including but not limited to antigen presenting cells, B-cells, basophils, cytotoxic T-cells, dendritic cells, eosinophils, granulocytes, helper T-cells, leukocytes, lymphocytes, macrophages, mast cells, memory cells, monocytes, natural killer cells, neutrophils, phagocytes, plasma cells and T-cells.

“Immune effector cell” as used herein refers to the T cells and natural killer (NK) cells.

“Immune response” as used herein refers to immunities including but not limited to innate immunity, humoral immunity, cellular immunity, immunity, inflammatory response, acquired (adaptive) immunity, autoimmunity and/or overactive immunity.

As used herein, “CD4 lymphocytes” refer to lymphocytes that express CD4, i.e., lymphocytes that are CD4+. CD4 lymphocytes may be T cells that express CD4.

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies.

The term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

“Single chain variable fragment”, “single-chain antibody variable fragments” or “scFv” antibodies as used herein refers to forms of antibodies comprising the variable regions of only the heavy (V_{H}) and light (V_{L}) chains, connected by a linker peptide. The scFvs are capable of being expressed as a single chain polypeptide. The scFvs retain the specificity of the intact antibody from which it is derived. The light and heavy chains may be in any order, for example, V_{H}-linker-V_{L} or V_{L}-linker-V_{H}, so long as the specificity of the scFv to the target antigen is retained.

“Therapeutic agents” as used herein refers to agents that are used to, for example, treat, inhibit, prevent, mitigate the effects of, reduce the severity of, reduce the likelihood of developing, slow the progression of and/or cure, a disease. Diseases targeted by the therapeutic agents include but are not limited to infectious diseases, carcinomas, sarcomas, lymphomas, leukemia, germ cell tumors, blastomas, antigens expressed on various immune cells, and antigens expressed on cells associated with various hematologic diseases, and/or inflammatory diseases.

“Cancer” and “cancerous” refers to or describe the physiological condition in mammals that is typically characterized by unregulated cell growth. The term “cancer” is meant to include all types of cancerous growths or oncogenic processes, metastatic tissues or malignantly transformed cells, tissues, or organs, irrespective of histopathologic type or stage of invasiveness. Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In one embodiment, the cancer is a melanoma, e.g., an advanced stage melanoma. Metastatic lesions of the aforementioned cancers can also be treated or prevented using the methods and compositions of the invention. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. Treatment of metastatic cancers, e.g., metastatic cancers that express PD-L1 (Iwai et al. (2005) Int. Immunol. 17:133-144) can be effected using the antibody molecules described herein.

The term “isolated” as used herein refers to molecules or biological materials or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both, cultured and engineered cells or tissues.

“Naked DNA” as used herein refers to DNA encoding a CAR cloned in a suitable expression vector in proper orientation for expression. Viral vectors which may be used include but are not limited SIN lentiviral vectors, retroviral vectors, foamy virus vectors, adeno-associated virus (AAV) vectors, hybrid vectors and/or plasmid transposons (for example sleeping beauty transposon system) or integrase based vector systems. Other vectors that may be used in connection with alternate embodiments of the invention will be apparent to those of skill in the art.

“Target cell” as used herein refers to cells which are involved in a disease and can be targeted by the genetically modified cells of the invention (including but not limited to genetically modified T-cells, NK cells, hematopoietic stem cells, pluripotent stem cells, and embryonic stem cells). Other target cells will be apparent to those of skill in the art and may be used in connection with alternate embodiments of the invention.

The terms “T-cell” and “T-lymphocyte” are interchangeable and used synonymously herein. Examples include but are not limited to naïve T cells, central memory T cells, effector memory T cells or combinations thereof.

“Vector”, “cloning vector” and “expression vector” as used herein refer to the vehicle by which a polynucleotide sequence (e.g. a foreign gene) can be introduced into a host cell, so as to transform the host and promote expression (e.g. transcription and translation) of the introduced sequence. Vectors include plasmids, phages, viruses, etc.

As used herein, the term “administering,” refers to the placement an agent as disclosed herein into a subject by a method or route which results in at least partial localization of the agents at a desired site.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. As non-limiting examples, “beneficial results” or “desired results” may be alleviation of one or more symptom(s), diminishment of extent of the deficit, stabilized (i.e., not worsening) state of cancer progression, delay or slowing of metastasis or invasiveness, and amelioration or palliation of symptoms associated with the cancer.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). In some embodiments, treatment of cancer includes decreasing tumor volume, decreasing the number of cancer cells, inhibiting cancer metastases, increasing life expectancy, decreasing cancer cell proliferation, decreasing cancer cell survival, or amelioration of various physiological symptoms associated with the cancerous condition.

“Conditions” and “disease conditions,” as used herein may include, cancers, tumors or infectious diseases. In exemplary embodiments, the conditions include but are in no way limited to any form of malignant neoplastic cell proliferative disorders or diseases. In exemplary embodiments, conditions include any one or more of kidney cancer, melanoma, prostate cancer, breast cancer, glioblastoma, lung cancer, colon cancer, or bladder cancer.

The term “effective amount” or “therapeutically effective amount” as used herein refers to the amount of a pharmaceutical composition comprising one or more peptides as disclosed herein or a mutant, variant, analog or derivative thereof, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the oligopeptides described herein. Measured or measurable parameters include clinically detectable markers of disease, for example, elevated or depressed levels of a biological marker, as well as parameters related to a clinically accepted scale of symptoms or markers for diabetes. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

CAR-T cells with antitumor activity are frequently exhausted in the immunosuppressive tumor microenvironment. The PD-1 receptor is a major effector in mediating T cell exhaustion. A previous study demonstrated that anti-PD-1 antibody treatment enhanced antitumor activity when combined with anti-HER2 CAR-T cells in a syngeneic breast carcinoma mouse model. However, achieving a substantial and sustained efficacy requires continuous administration and a large amount of antibodies, often leading to severe systemic toxicity. Therefore, instead of administering the anti-PD-1 antibody systemically, we engineered anti-PD-1 self-secreting CAR.αPD1-T cells, which are less exhausted, more functional and expandable, and more efficient at mediating tumor eradication compared to injection of CAR-T cells alone, or the combined injection of anti-PD-1 antibody with the CAR-T cells. Our study provides an efficient and safe strategy for combining CPI treatment with CAR-T cell therapy for immunotherapy in solid tumors.

Accordingly, provided herein is a cell (for example, a genetically modified cell) containing a nucleic acid encoding both a chimeric antigen receptor (CAR) and a checkpoint inhibitor, or nucleic acids encoding a CAR and a CPI, respectively. In various embodiments, the cell expresses a CAR and a checkpoint inhibitor. In one embodiment, the cell is a lymphocyte cell (T-cell). In one embodiment, the cell is a Natural Killer (NK) cells. In various embodiments, the checkpoint inhibitor (for example, anti-PD-1 scFv) is constitutively expressed.

In some embodiments, the cell (for example, a genetically modified cell) expresses a CAR that targets any one or more of targets expressed on disease causing or disease associated cells including but not limited to CD19, CD22, CD23, MPL, CD30, CD32, CD20, CD70, CD79b, CD99, CD123, CD138, CD179b, CD200R, CD276, CD324, FcRH5, CD171, CS-1, CLL-1 (CLECL1), CD33, CDH1, CDH6, CDH16, CDH17, CDH19, EGFRviii, FcRH5, GD2, GD3, HLA-A2, BCMA, Tn Ag, PSMA, ROR1, FLT3, FAP, TAG72, CD38, CD44v6, CEA, EPCAM, B7H3, KIT, IL-13Ra2, IL11Ra, Mesothelin, PSCA, VEGFR2, Lewis Y, CD24, PDGFR-beta, PRSS21, SSEA-4, CD20, Fc region of an immunoglobulin, Tissue Factor, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLea, GM3, TGS5, HMWMAA, o-acetyl-GD2, Folate receptor beta, TEM1/CD248, TEM7R, CLDN6, TSHR, TCR-beta1 constant chain, TCR beta2 constant chain, TCR gamma-delta, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, HTLV1-Tax, KSHV K8.1 protein, EBB gp350, HIV1-envelop glycoprotein gp120, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, DLL3, TROP2, PTK7, GCC, AFP, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, or combinations thereof.

In one embodiment, the cell (for example, a genetically modified cell) expresses a CAR that targets CD19.

In some embodiments, the cell (for example, a genetically modified cell) expresses a checkpoint inhibitor target any one or more of PD-1, LAG-3, TIM3, B7-H1, CD160, P1H, 2B4, CEACAM (e.g., CEACAM-1, CEACAM-3, and/or CEACAM-5), TIGIT, CTLA-4, BTLA, and LAIR1. In some embodiments, the checkpoint inhibitors are antibodies or fragments thereof that target any one or more of PD-1, LAG-3, TIM3, B7-H1, CD160, P1H, 2B4, CEACAM (e.g., CEACAM-1, CEACAM-3, and/or CEACAM-5), CTLA-4, BTLA, and LAIR1.

In one embodiment, the cell (for example, a genetically modified cell) expresses the checkpoint inhibitor that targets PD-1. In one embodiment, the checkpoint inhibitor is an anti-PD-1 scFv.

In one embodiment, the cell (for example, a genetically modified cell) expresses a CAR that targets CD19 and a checkpoint inhibitor that targets PD-1, wherein the checkpoint inhibitor that targets PD-1 is an anti-PD-1-scFv.

Also provided herein is a nucleic acid comprising a first polynucleotide encoding the CAR described herein and a second polynucleotide encoding the checkpoint inhibitor described herein. Also provided herein are polypeptides encoded by the one or more nucleic acids described herein. Further provided herein is a vector comprising the one or more nucleic acids described herein.

Further provided herein are methods for treating, inhibiting, preventing metastasis of, and/or reducing the severity of cancer in a subject in need thereof. The methods comprise administering to a subject in need thereof, a therapeutically effective amount of a cell comprising a nucleic acid encoding a chimeric antigen receptor and a checkpoint inhibitor (or nucleic acids encoding a CAR and a CPI, respectively), so as to treat, inhibit, prevent metastasis of and/or reduce severity of cancer in the subject. In an exemplary embodiment, the cancer is lung cancer.

Further provided herein are methods for treating, inhibiting, preventing metastasis of, and/or reducing the severity of cancer in a subject in need thereof. The methods include administering a therapeutically effective amount of a composition including a cell that contains a nucleic acid encoding both a chimeric antigen receptor (CAR) and a checkpoint inhibitor, or a cell that contains nucleic acids encoding a CAR and a checkpoint inhibitor, respectively, to the subject so as to treat, inhibit, prevent metastasis of and/or reduce severity of cancer in the subject. In an exemplary embodiment, the cancer is lung cancer.

Further provided herein are methods for treating, inhibiting, preventing metastasis of, and/or reducing the severity of lung cancer in a subject in need thereof. The methods comprise administering a therapeutically effective amount of a composition comprising a cell comprising a nucleic acid encoding both a CD19 specific chimeric antigen receptor and a PD-1 specific checkpoint inhibitor (for example, anti-PD-1-scFv), or nucleic acids encoding a CD19 specific CAR and a PD-1 specific checkpoint inhibitor, respectively, to the subject so as to treat, inhibit, prevent metastasis of and/or reduce severity of lung cancer in the subject.

In various embodiments, the methods further comprise administering the subject a therapeutically effective amount of existing therapies (existing therapeutic agents), wherein the existing therapies are administered sequentially or simultaneously with the compositions described herein.

In some embodiments, the cells (genetically modified cells) described herein may be used in a treatment regimen in combination with existing therapies including but not limited to surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al. 2008 J Neurosurg 108:963-971. In one embodiment, a CAR-expressing cell described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an anti metabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

When a “therapeutically effective amount” is indicated, the precise amount of the compositions of the present invention to be administered can be determined by a physician with consideration of individual differences in age, weight, tumor size, extent of infection or metastasis, and condition of the patient (subject). In some embodiments, the therapeutically effective amount of the genetically modified cells is administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The cells can be administered by injection into the site of the lesion (e.g., intra-tumoral injection).

In one embodiment, the CAR and CPI are introduced into immune effector cells (e.g., T cells, NK cells), e.g., using in vitro transcription, and the subject (e.g., human) receives an initial administration of the immune effector cells (e.g., T cells, NK cells) comprising the CAR and CPI of the invention, and one or more subsequent administrations of the immune effector cells (e.g., T cells, NK cells) comprising the CAR and CPI of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the immune effector cells (e.g., T cells, NK cells) comprising the CAR and CPI of the invention are administered to the subject (e.g., human) per week, e.g., 2, 3, or 4 administrations of the immune effector cells (e.g., T cells, NK cells) comprising the CAR and CPI of the invention are administered per week. In one embodiment, the subject (e.g., human subject) receives more than one administration of the immune effector cells (e.g., T cells, NK cells) comprising the CAR and CPI of the invention per week (e.g., 2, 3 or 4 administrations per week) (also referred to herein as a cycle), followed by a week of no immune effector cells (e.g., T cells, NK cells) administrations, and then one or more additional administration of the immune effector cells (e.g., T cells, NK cells) comprising the CAR and CPI of the invention (e.g., more than one administration of the immune effector cells (e.g., T cells, NK cells) per week is administered to the subject. In another embodiment, the subject (e.g., human subject) receives more than one cycle of immune effector cells (e.g., T cells, NK cells) comprising the CAR and CPI, and the time between each cycle is less than 10, 9, 8, 7, 6, 5, 4, or 3 days. In one embodiment, the immune effector cells (e.g., T cells, NK cells) comprising the CAR and CPI are administered every other day for 3 administrations per week. In one embodiment, the immune effector cells (e.g., T cells, NK cells) comprising the CAR and CPI of the invention are administered for at least two, three, four, five, six, seven, eight or more weeks.

In some embodiments, the therapeutic methods described herein further comprise administering to the subject, sequentially or simultaneously, existing therapies. Examples of existing cancer treatment include, but are not limited to, active surveillance, observation, surgical intervention, chemotherapy, immunotherapy, radiation therapy (such as external beam radiation, stereotactic radiosurgery (gamma knife), and fractionated stereotactic radiotherapy (FSR)), focal therapy, systemic therapy, vaccine therapies, viral therapies, molecular targeted therapies, or combinations thereof.

In some embodiments, methods for preparing the genetically modified cells (containing one or more nucleic acid encoding one or more CARs and one or more CPIs as described herein) include obtaining a population of cells and selecting cells that express any one or more of CD3, CD28, CD4, CD8, CD45RA, and/or CD45RO. In certain embodiments, the population of immune effector cells provided are CD3+ and/or CD28+.

In one embodiment, the method for preparing the genetically modified cells (containing one or more nucleic acid encoding one or more CARs and one or more CPIs as described herein) include obtaining a population of cells and enriching for the CD25+ T regulatory cells, for example by using antibodies specific to CD25. Methods for enriching CD25+ T regulatory cells from the population of cells will be apparent to a person of skill in the art. In some embodiments, the Treg enriched cells comprise less than 30%, 20%, 10%, 5% or less non-Treg cells. In some embodiments, the vectors encoding the CARs and CPIs described herein are transfected into Treg-enriched cells. Treg enriched cells expressing a CAR and a CPI may be used to induced tolerance to antigen targeted by the CAR.

In some embodiments, the method further includes expanding the population of cells after the vector(s) comprising nucleic acid(s) encoding the CARs and CPIs described herein have been transfected into the cells. In embodiments, the population of cells is expanded for a period of 8 days or less. In certain embodiments, the population of cells is expanded in culture for 5 days, and the resulting cells are more potent than the same cells expanded in culture for 9 days under the same culture conditions. In other embodiments, the population of cells is expanded in culture for 5 days show at least a one, two, three or four fold increase in cell doublings upon antigen stimulation as compared to the same cells expanded in culture for 9 days under the same culture conditions. In some embodiments, the population of cells is expanded in an appropriate media that includes one or more interleukins that result in at least a 200-fold, 250-fold, 300-fold, or 350-fold increase in cells over a 14 day expansion period, as measured by flow cytometry.

In various embodiments, the expanded cells comprise one or more CARs and one or more CPIs as described herein.

### Pharmaceutical Composition

In various embodiments, the present invention provides a pharmaceutical composition. The pharmaceutical composition includes a cell comprising nucleic acids encoding a CAR and a checkpoint inhibitor, as described herein. The pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the rAAV vector, the cell transfected with the rAAV vector, or the supernatant conditioned with the transfected cell. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

### Kits

In various embodiments, the present invention provides a kit for treating cancer comprising a composition that includes cells comprising nucleic acids encoding one or more CARs and one or more CPIs, as described herein.

The kit is an assemblage of materials or components, including at least one of the inventive compositions (for example, genetically modified cells comprising nucleic acids encoding one or more CARs and one or more CPIs, as described herein). Thus, in some embodiments the kit contains a composition including a drug delivery molecule complexed with a therapeutic agent, as described above.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat, reduce the severity of, inhibit cancer in a subject. Still in accordance with the present invention, “instructions for use” may include a tangible expression describing the preparation of the composition and/or at least one method parameter, such as the relative amounts of composition, dosage requirements and administration instructions, and the like, typically for an intended purpose. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition containing a volume of the AAV1-P0-ICE vector. The packaging material generally has an external label which indicates the contents and/or purpose of the kit and/or its components.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

### Experimental Methods

Mice. Six- to eight-week-old female NOD.Cg-Prkdc^{scid}IL2Rg^{tm1Wj1}.Sz (NSG) mice were purchased from Jackson Laboratory (Farmington, Conn.). All animal studies were performed in accordance with the Animal Care and Use Committee guidelines of the NIH and were conducted under protocols approved by the Animal Care and Use Committee of the NCI.

Cell culture and antibodies. Cell lines SKOV3 and 293T were obtained from ATCC. The lung cancer line NCI-H292 was kindly provided by Dr. Ite Laird-Offringa (University of Southern California, Los Angeles, Calif.). The H292-CD19 and SKOV3-CD19 cell lines were generated by the transduction of parental NCI-H292 and SKOV3 cells with a lentiviral vector encoding the cDNA of human CD19. The transduced H292 and SKOV3 cells were stained with anti-human CD19 antibody (BioLegend, San Diego, Calif.) and sorted to yield a relatively pure population of CD19-overexpressing cells. SKOV3, SKOV3-CD19, NCI-H292, and H292-CD19 cells were maintained in R10 medium consisting of RPMI-1640 medium supplemented with 10% fetal bovine serum (FBS), 2 mM L-glutamine, 10 mM HEPES, 100 U/ml penicillin and 100 μg/ml streptomycin. The 293T cells were cultured in D10 medium consisting of DMEM medium supplemented with 10% FBS, 2 mM L-glutamine, 10 mM HEPES, 100 U/ml penicillin and 100 μg/ml streptomycin. All above cell culture media and supplements were purchased from Hyclone (Logan, Utah). Human peripheral blood mononuclear cells (PBMCs) were cultured in T cell medium (TCM), which is composed of X-Vivo 15 medium (Lonza, Walkersville, Md.) supplemented with 5% human AB serum (GemCell, West Sacramento, Calif.), 1% HEPES (Gibco, Grand Island, N.Y.), 1% Pen-Strep (Gibco), 1% GlutaMax (Gibco), and 0.2% N-Acetyl Cysteine (Sigma-Aldrich, St. Louis, Mo.).

Primary antibodies used in this study include biotinylated Protein L (GeneScript, Piscataway, N.J.); PE-anti-CD45, PE-Cy5.5-anti-CD3, FITC-anti-CD4, Pacific Blue™-anti-CD8, FITC-anti-CD8, PE-anti-IFN-γ, Brilliant Violet 421™-anti-PD-1, PE-anti-PD-L1, PerCP/Cy5.5-anti-LAG-3, and PE-anti-TIM-3 (BioLegend, San Diego, Calif.); and Rabbit anti-HA tag antibody (Abeam, Cambridge, Mass.). The secondary antibodies used were FITC-conjugated streptavidin (BioLegend, San Diego, Calif.) and goat anti-rabbit IgG-HRP (Santa Cruz, San Jose, Calif.). The SuperSignal® West Femto Maximum Sensitivity Substrate used for Western blot analysis was from Thermo Fisher Scientific (Waltham, Mass.).

Plasmid construction. The retroviral vector encoding anti-CD19 CAR (CAR) was constructed based on the MP71 retroviral vector kindly provided by Prof. Wolfgang Uckert, as described previously (Engels B, et al. 2003. Retroviral vectors for high-level transgene expression in T lymphocytes. Hum Gene Ther 14: 1155-68. The vector encoding anti-CD19 CAR with anti-PD-1 scFv (CAR.αPD1) was then generated based on the anti-CD19 CAR. The insert for CAR.αPD1 vector consisted of the following components in frame 5′ end to 3′ end: the anti-CD19 CAR, an EcoRI site, a leader sequence derived from human IL-2, the anti-PD-1 scFv light chain variable region, a GS linker, the anti-PD-1 scFv heavy chain variable region, the HA-tag sequence, and a NotI site.

The anti-PD-1 scFv portion in the CAROM vector was derived from the amino acid sequence of human monoclonal antibody 5C4 specific against human PD-1 (Alan J. Korman M S, Changyu Wang, Mark J. Selby, Bingliana Chen, Josephine M. Cardarelli. 2011. United States. The corresponding DNA sequence of the scFv was codon-optimized for its optimal expression in human cells using the online codon optimization tool and was synthesized by Integrated DNA Technologies (Coralville, Iowa). The anti-PD-1 scFv was then ligated into the CD19 CAR vector via the EcoRI site through the Gibson assembly method.

Retroviral vector production. Retroviral vectors were prepared by transient transfection of 293T cells using a standard calcium phosphate precipitation protocol. 293T cells cultured in 15-cm tissue culture dishes were transfected with 37.5 μg of the retroviral backbone plasmid, along with 18.75 μg of the envelope plasmid pGALV and 30 μg of the packaging plasmid encoding gag-pol. The viral supernatants were harvested 48 h post-transfection and filtered through a 0.45 μm filter (Corning, Corning, N.Y.) before use.

T cell transduction and expansion. Frozen human PBMCs were obtained from AllCells (Alameda, Calif.). PBMCs were thawed in TCM and rested overnight. Before retroviral transduction, PBMCs were activated for 2 days by culturing with 50 ng/ml OKT3, 50 ng/ml anti-CD28 antibody, and 10 ng/ml recombinant human IL-2 (PeproTech, Rocky Hill, N.J.). For transduction, freshly harvested retroviral supernatant was spin-loaded onto non-tissue culture-treated 12-well plates coated with 15 μg retronectin (Clontech Laboratories, Mountain View, Calif.) per well by centrifuging 2 hours at 2000×g at 32° C. The spin-loading of vector was repeated once with fresh viral supernatant. Activated PBMCs were resuspended at the concentration of 5×10⁵ cells/ml with fresh TCM complemented with 10 ng/ml recombinant human IL-2 and added to the vector-loaded plates. The plates were spun at 1000×g at 32° C. for 10 minutes and incubated overnight at 37° C. and 5% CO₂. The same transduction procedure was repeated on the following day. During ex vivo expansion, culture medium was replenished, and cell density was adjusted to 5×10⁵/ml every two days.

Surface immunostaining and flow cytometry. To detect anti-CD19 CAR expression on the cell surface, cells were stained with protein L. Before FACS staining, 5×10⁵ cells were harvested and washed three times with FACS buffer (PBS containing 5% bovine serum albumin fraction V). Cells were then stained with 1 μg of biotinylated protein L at 4° C. for 30 minutes. Cells were washed with FACS buffer three times and then incubated with 0.1 μg of FITC-conjugated streptavidin in FACS buffer at 4° C. for 10 minutes. Cells were washed and fixed with TransFix cellular antigen stabilizing reagent (Thermo Scientific, Waltham, Mass.) at 4° C. for 10 minutes. Cells were then washed twice and stained with anti-CD3, anti-CD4, and anti-CD8 at 4° C. for 10 minutes. Cells were washed and resuspended in PBS. Fluorescence was assessed using a MACSquant cytometer (Miltenyi Biotec, San Diego, Calif.), and all the FACS data were analyzed using FlowJo software (Tree Star, Ashland, Oreg.).

Intracellular cytokine staining. T cells (1×10⁶) were cultured with target cells at a ratio of 1:1 for 6 hours at 37° C. and 5% CO₂ with GolgiPlug (BD Biosciences, San Jose, Calif.) in 96-well round bottom plates. PE-Cy5.5-anti-CD3, FITC-anti-CD4, Pacific blue-CD8, PE-anti-IFN-γ and PE-anti-Ki67 antibodies were used for the intracellular staining. Cytofix/Cytoperm Fixation and Permeabilization Kit (BD Biosciences) was used to permeabilize the cell membrane and perform intracellular staining according to the manufacturer's instruction.

Western blotting analysis. Cell culture supernatant was harvested, and anti-PD-1 scFv was purified with Pierce™ anti-HA magnetic beads (Thermo Scientific, Waltham, Mass.) according to the manufacturer's instruction. The purified antibody was then subjected to SDS-PAGE, and transferred to a nitrocellulose membrane (Thermo Scientific, Waltham, Mass.) for Western blot analysis. The Western blot was analyzed with anti-HA tag antibody (Abcam, Cambridge, Mass.) as described previously (Xu S et al. 2012. Discovery of an orally active small-molecule irreversible inhibitor of protein disulfide isomerase for ovarian cancer treatment. Proc Natl Acad Sci USA 109: 16348-53).

ELISA. IFN-γ was measured using a human IFN-γ ELISA kit (BD Biosciences, San Jose, Calif.) according to the manufacturer's instructions. Briefly, 96-well ELISA plates (Thermo Scientific, Waltham, Mass.) were coated with 200 ng/well of capture antibodies against the indicated proteins at 4° C. overnight. On the next day, plates were washed with wash buffer (PBS containing 0.05% Tween 20) and blocked with assay buffer (PBS containing 10% FBS) for 2 hours at room temperature. Equal volume of serum, or cell culture supernatant was added to the plate and incubated for 2 hours at room temperature. Plates were then washed and incubated with detection antibodies for 1 hour at room temperature. To measure anti-PD-1 antibody and secreted anti-PD-1 say, recombinant human PD-1 (rhPD-1) was used to pre-coat the plate. Goat anti-mouse IgG1-HRP and anti-HA tag antibodies were used as detection antibodies, respectively.

Competitive blocking assay. The 96-well assay plates (Thermo Scientific, Waltham, Mass.) were coated with 3 μg/ml of anti-human CD3 antibody at 4° C. overnight. On the second day, the supernatant of the wells was aspirated and the wells were washed once with 100 μl per well of PBS. 10 μg/ml of rhPD-L1/Fc (R&D Systems, Minneapolis, Minn.) in 100 μl of PBS were added. In each well, 100 μg/ml of goat anti-human IgG Fc antibody in 10 μl of PBS were then added. The assay plate was incubated for 4 hours at 37° C. Human T cells were harvested, washed once and then resuspended to 1×10⁶ cells/ml in TCM. The wells of the assay plate were aspirated. Then, 100 μl of human T-cell suspension (1×10⁵) and 100 μl of supernatant of CAR or CAR.αPD1 T cell culture 3-day post-transduction, supplemented with GolgiPlug (BD Biosciences), were added to each well. The plate was covered and incubated at 37° C. and 5% CO₂ overnight. After incubation, T cells were harvested and stained with IFN-γ intracellularly.

Specific cell lysis assay. Lysis of target cells (H292-CD19) was measured by comparing the survival of target cells to the survival of the negative control cells (NCI-H292). This method has been described previously (Kochenderfer J N, et al 2009. Construction and preclinical evaluation of an anti-CD19 chimeric antigen receptor. J Immunother 32: 689-702). NCI-H292 cells were labeled by suspending them in R10 medium with 5 μM CellTracker Orange (5-(and-6)-(((4-chloromethyl)benzoyl)amino)tetramethylrhodamine) (CMTMR), a fluorescent dye for monitoring cell movement (Invitrogen, Carlsbad, Calif.), at a concentration of 1.5×10⁶ cells/mL. The cells were incubated at 37° C. for 30 minutes and then washed twice and suspended in fresh R10 medium. H292-CD19 cells were labeled by suspending them in PBS+0.1% BSA with 5 μM Carboxyfluorescein succinimidyl ester (CFSE) fluorescent dye at a concentration of 1×10⁶ cells/mL. The cells were incubated for 30 minutes at 37° C. After incubation, the same volume of FBS was added into the cell suspension and then incubated for 2 minutes at room temperature. The cells were then washed twice and suspended in fresh R10 medium. Equal amounts of NCI-H292 and H292-CD19 cells (5×10⁴ each) were combined in the same well for each culture with effector CAR-T cells. Cocultures were set up in round bottom 96-well plates in triplicate at the following effector-to-target ratios: 1:1 and 5:1. The cultures were incubated for 4 hours at 37° C., followed by 7-AAD labeling, according to the manufacturer's instructions (BD Biosciences). Flow cytometric analysis was performed to quantify remaining live (7-AAD-negative) target cells. For each coculture, the percent survival of H292-CD19 cells was determined by dividing the percentage of live H292-CD19 cells by the percentage of live NCI-H292 cells. In the wells containing only target and negative control cells without effector cells, the ratio of the percentage of H292-CD19 cells to the percentage of NCI-H292 cells was calculated and used to correct the variation in the starting cell numbers and spontaneous cell death. The cytotoxicity was determined in triplicate and presented in mean±SEM.

Cell proliferation. 3×10⁵ H292-CD19 cells were suspended in D10 medium and then seeded in a 6-well plate. Once the target cells attached, nontransduced T cells, CAR and CAR.aPD1 T cells were harvested and washed twice with PBS. The cells were then labeled by suspending them in PBS with 10 μM CFSE at a concentration of 1×10⁶ cells/mL and incubated for 60 minutes at 37° C. After incubation, the cells were washed twice and suspended in fresh TCM. An equal number of T cells were added to the target cells for coculture. Cocultures were set up in triplicate at an effector-to-target ratio of 1:1. The cultures were incubated for 96 hours at 37° C. Flow cytometric analysis was performed to quantify the intensity of CFSE on T cells. The proliferation rates were determined in triplicate and presented in mean±SEM.

Tumor model and adoptive transfer. At 6 to 8 weeks of age, mice were inoculated subcutaneously with 3×10⁶ H292-CD19 cells, and 10-13 days later, when the average tumor size reached 100-120 mm³, mice were treated with i.v. adoptive transfer of 1×10⁶ or 3×10⁶ CAR transduced T cells in 100 μl PBS. CAR expression was normalized to 20% in both CAR groups by addition of donor-matched nontransduced T cells. Tumor growth was monitored twice a week. Tumor size was measured by calipers and calculated by the following formula: W²×L/2. Mice were euthanized when they displayed obvious weight loss, ulceration of tumors, or tumor size larger than 1000 mm³.

Statistical analysis. Statistical analysis was performed in GraphPad Prism, version 5.01. One-way ANOVA with Tukey's multiple comparison was performed to assess the differences among different groups in the in vitro assays. Tumor growth curve was analyzed using one-way ANOVA with repeated measures (Tukey's multiple comparison method). Mouse survival curve was evaluated by the Kaplan-Meier analysis (log-rank test with Bonferroni correction). A P value less than 0.05 was considered statistically significant. Significance of findings was defined as: ns=not significant, P>0.05; *, P<0.05; **, P<0.01; ***, P<0.001.

### Characterization of Anti-CD19 CAR-T Cells Secreting Anti-PD-1 Antibody

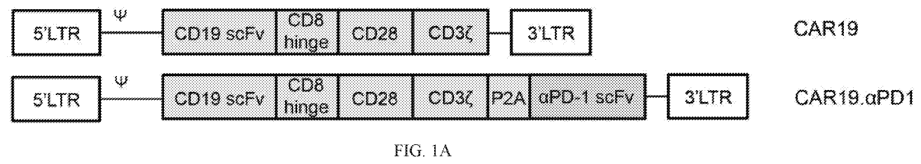

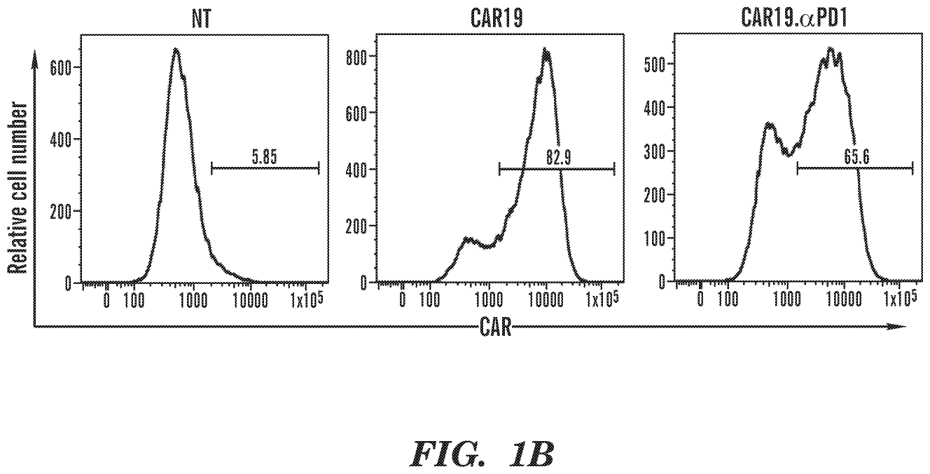

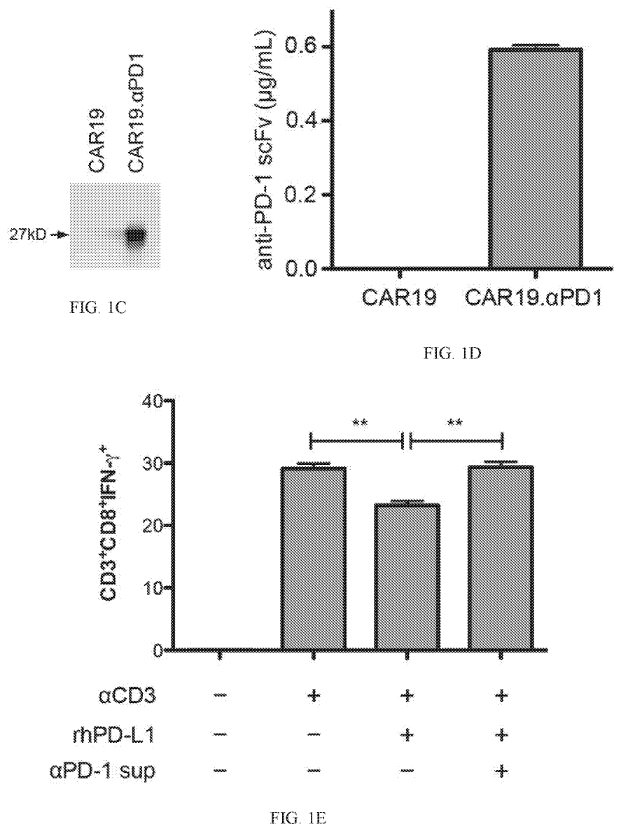

The schematic representation of the retroviral vector constructs used in this study is shown in FIG. 1A. The retroviral vector encoding the anti-CD19 CAR composed of anti-CD19 scFv, CD8 hinge, CD28 transmembrane and intracellular costimulatory domains, as well as intracellular CD35 domain was designated as CAR19. The retroviral vector encoding both anti-CD19 CAR and secreting anti-PD-1 scFv was designated as CAR19.αPD1. Human PBMCs were transduced with each construct to test the expression of CAR in primary lymphocytes. As seen in FIG. 1B, CAR expression was observed for both constructs in human T cells, although anti-PD-1-secreting CAR19 T cells expressed slightly lower level of the CAR on the cell surface. Expression and secretion of anti-PD-1 was assessed by performing Western blotting analysis and ELISA on the cell supernatant three days post-transduction. We observed that anti-PD-1 could be successfully expressed and secreted by T cells transduced with CAR19.αPD1 (FIG. 1C and FIG. 1D).

To evaluate the binding activity and blocking function of anti-PD-1 scFv secreted by CAR19.αPD1 T cells, a competitive binding and blocking assay was performed. Intracellular IFN-γ was measured to assess the activity of the T cells. As shown in FIG. 1E, the expression of IFN-γ was upregulated when the T cells were stimulated by anti-CD3 antibody, whereas the presence of recombinant human PD-L1 (rhPD-L1) resulted in significantly lower IFN-γ expression. However, adding the cell culture supernatant from CAR19.αPD1 T cells effectively reversed the inhibitory effect of rhPD-L1 on the T cells and significantly increased IFN-γ production (FIG. 1E).

### Secreting Anti-PD-1 Antibody Enhances the Antigen-Specific Immune Responses of CAR-T Cells

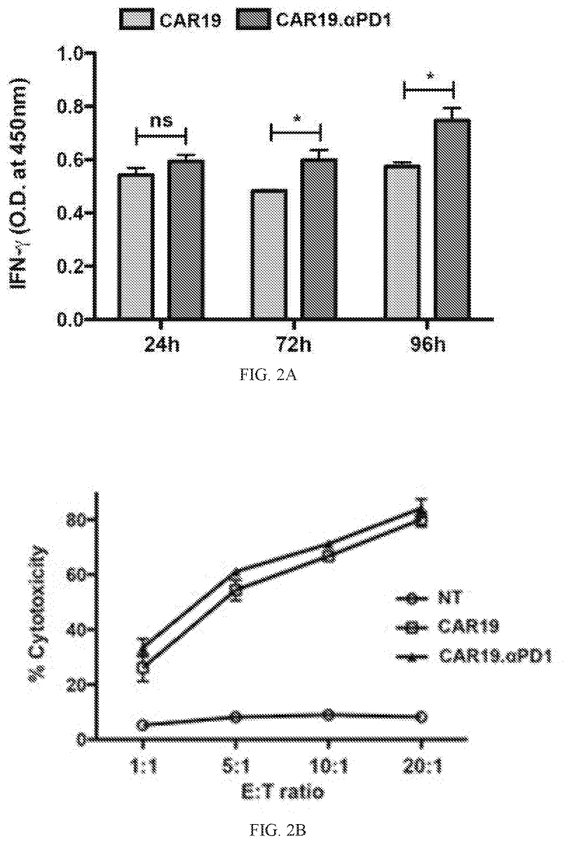

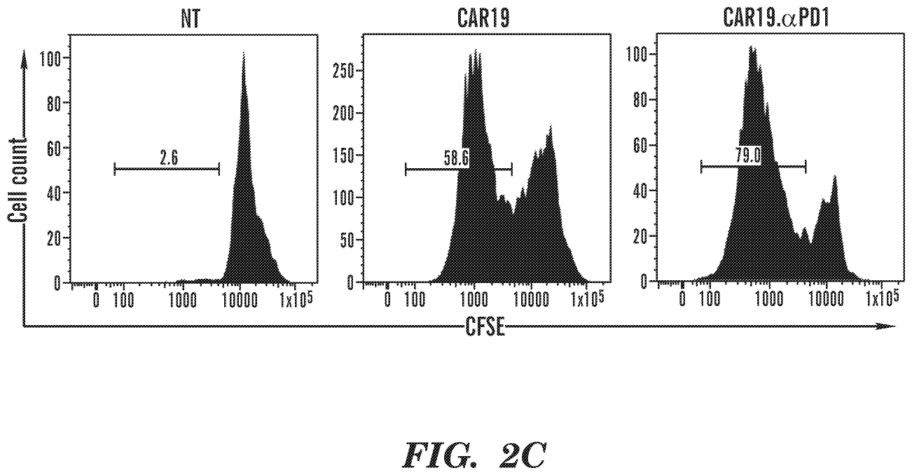

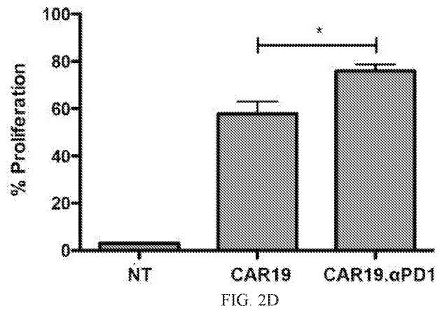

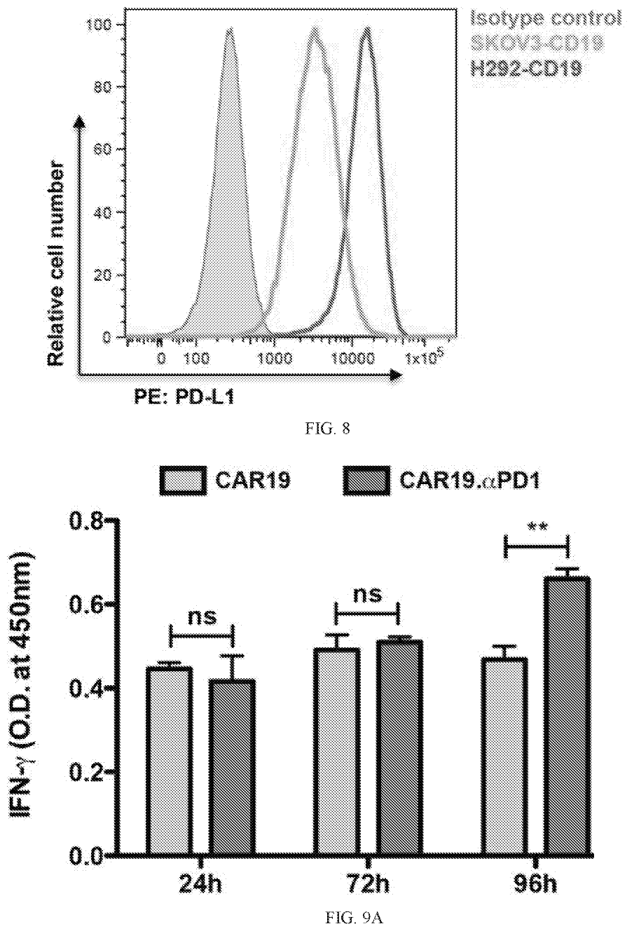

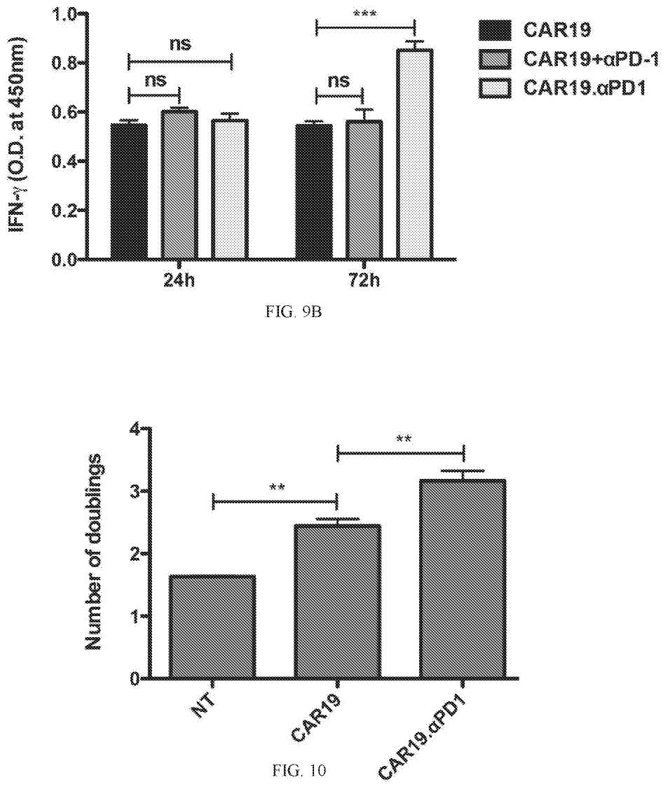

To further assess the effector function of anti-PD-1-secreting CAR19 T cells through antigen-specific stimulation, both CAR19 and CAR19.αPD1 T cells were cocultured for different durations with H292-CD19 or SKOV3-CD19 target cells, both of which were shown to have high surface expression of PD-L1 (FIG. 8). T cells at different time points were then harvested, and the cell function marker IFN-γ in the supernatant was measured by ELISA. Upon antigen stimulation for 24 hours, we found that both CAR19 and CAR19.αPD1 T cells, with or without secreting anti-PD-1, had a similar amount of IFN-γ secretion (FIG. 2A and FIG. 9A). However, after 72 hours, CAR19.αPD1 T cells secreted significantly higher IFN-γ compared to the parental CAR19 T cells after stimulation with H292-CD19 cells (FIG. 2A). Similarly, after 96 hours of antigen stimulation, CAR19 T cells secreting anti-PD-1 expressed significantly more IFN-γ than that expressed by the parental CAR19 T cells (FIG. 2A and FIG. 9A).

Next, the cytolytic function of engineered T cells was examined by a 6-hour cytotoxicity assay. The cytotoxic activity of CAR19 and CAR19.αPD1 T cells against H292-CD19 cells was evaluated at effector/target (E/T) ratios of 1, 5, 10 and 20. We found that both CAR19 and CAR19.αPD1 T cells mediated significant cell lysis of target cells, especially at higher E/T ratios in comparison with the nontransduced T cells. However, little difference was found between CAR19 and CAR19.αPD1 T cells in terms of cytolytic activity (FIG. 2B). T cell proliferation was then evaluated by a carboxyfluorescein diacetate succinimidyl ester (CFSE)-based proliferation assay after 96-hour coculture of engineered T cells with target H292-CD19 cells. We observed that antigen-specific stimulation of both CAR19 and CAR19.αPD1 T cells resulted in a markedly higher level of proliferation compared to nontransduced T cells. Moreover, compared to CAR19 T cells (57.9±10.2%), the proliferation rate of CAR19.αPD1 T cells (75.9±5.5%) was significantly higher (FIG. 2C and FIG. 2D). The cell proliferation potential was further assessed by cell expansion. With antigen-specific stimulation, it was shown that both CAR19 and CAR19.αPD1 T cells significantly expanded compared to the nontransduced T cells. Remarkably, in comparison with parental CAR19 T cells (2.4±0.2), the number of cell doublings was significantly higher in CAR19.αPD1 T cells (3.2±0.3) (FIG. 10).

### Secreting Anti-PD-1 Alleviates CAR T Cell Exhaustion After Antigen Stimulation

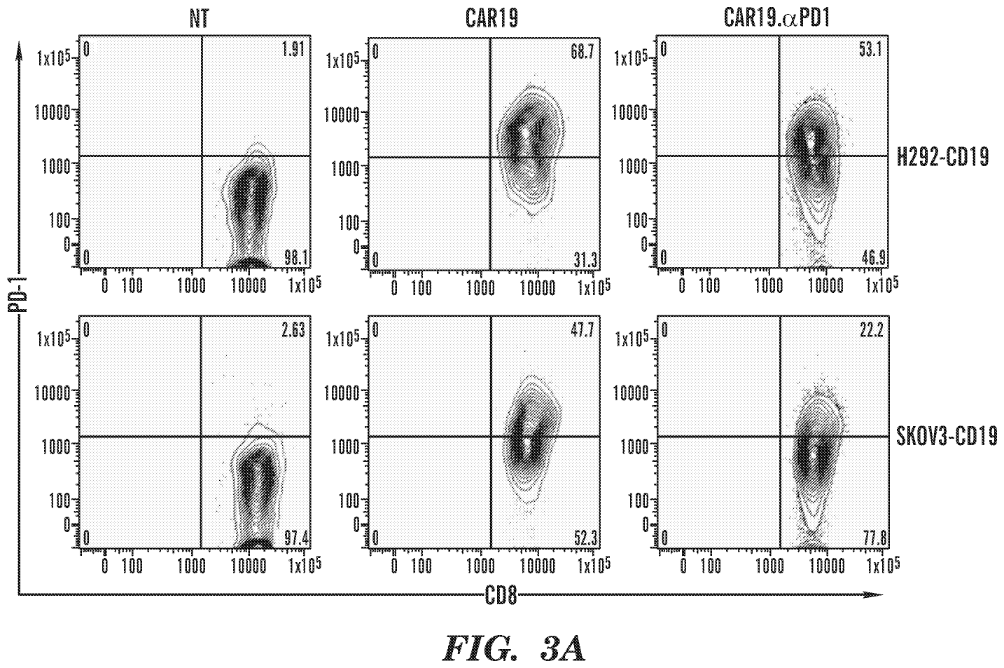

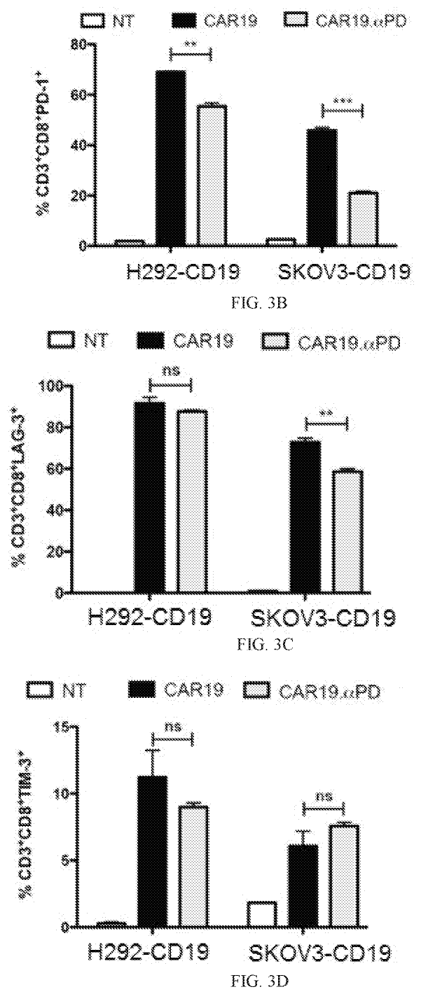

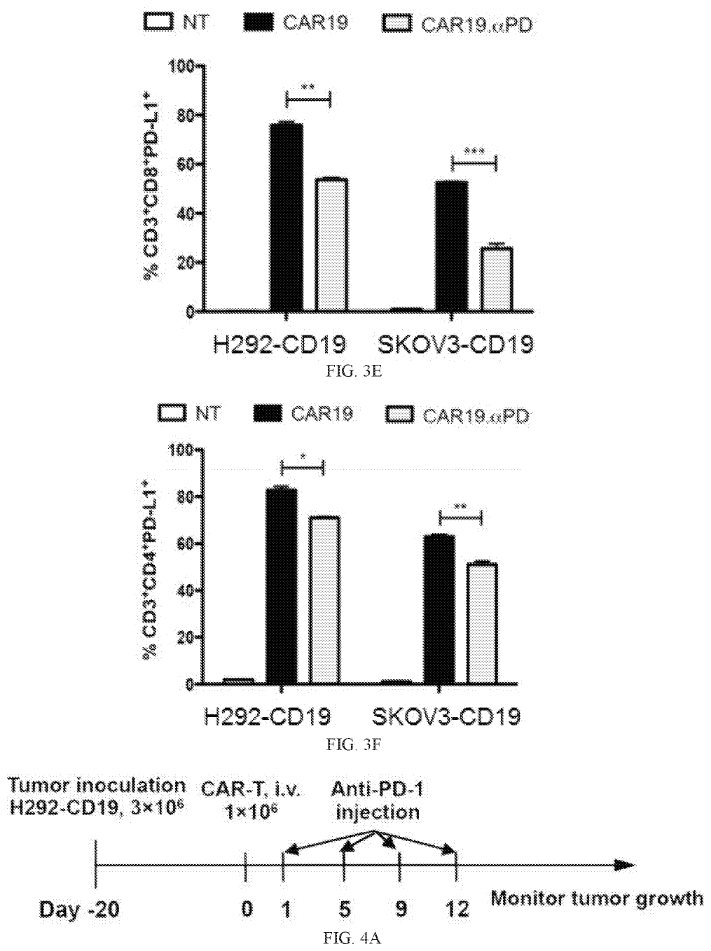

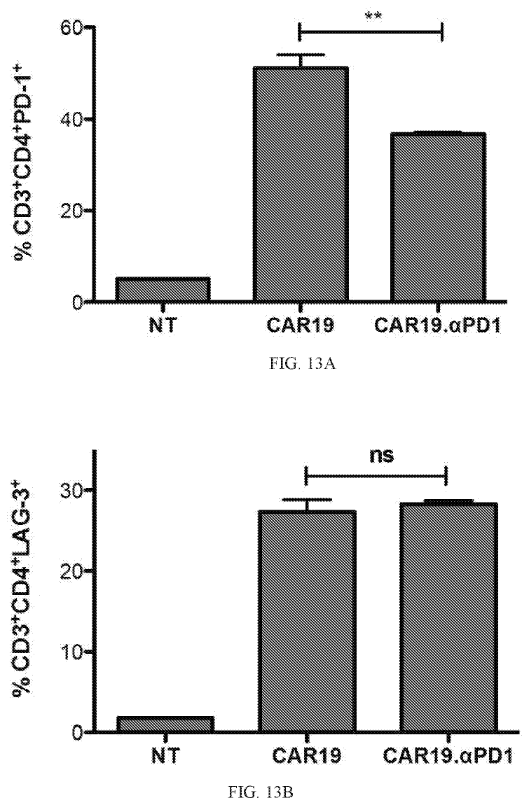

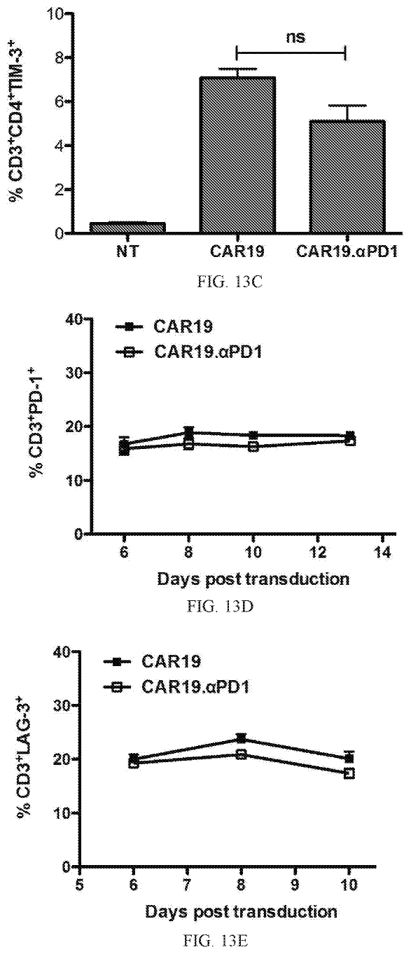

PD-1 expression on human GD2 and mouse HER2 CAR T cells has been shown to increase following antigen-specific activation, and PD-1 blockade was found to downregulate PD-1 expression in T cells. To assess the effect of secreted anti-PD-1 scFv on protecting human T cells from exhaustion, the engineered CAR T cells were cocultured with either H292-CD19 or SKOV3-CD19 target cells for 24 hours and then stained for the T cell exhaustion marker PD-1. We found that the expression of PD-1 was significantly upregulated in both CAR19 and CAR19.αPD1 T cells following antigen-specific stimulation. In comparison, the upregulated PD-1 expression on CAR19.αPD1 T cells was significantly lower than that on parental CAR19 T cells (FIG. 3A, FIG. 3B, and FIGS. 13A-13C). However, without antigen-specific stimulation, the expression of PD-1 in both CAR19 and CAR19.αPD1 T cells maintained at a similar and stable level over the course of T cell expansion (FIGS. 13D and 13E).

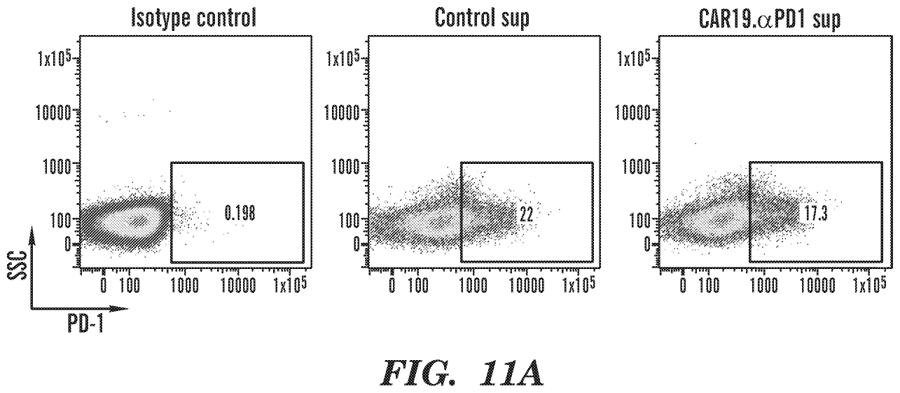

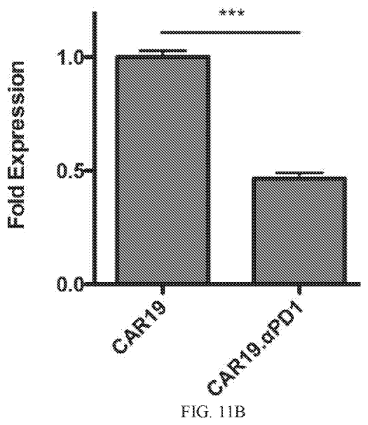

To further determine whether the lower expression of PD-1 in CAR19.αPD1 T cells is due to the blocking function of secreted anti-PD-1 scFv on the binding of PD-1 detection antibody or the downregulation of PD-1, we incubated the activated T cells with either the control medium or CAR19.αPD1 T cell culture supernatant for 30 min before staining them with anti-PD-1 antibody. We found that the secreted anti-PD-1 scFv was able to block approximately 20% of the binding of the PD-1 detection antibody (FIG. 11A). In tandem, we cocultured either the CAR19 or CAR19.αPD1 T cells with target cells H292-CD19 for 24 hours. Both T cells were then harvested and the transcriptional expression of PD-1 was measured by q-PCR. We observed that PD-1 expression in CAR19.αPD1 T cells was significantly lower than that in parental CAR19 T cells (FIG. 11B). This indeed confirms that CAR19.αPD1 T cells have downregulated PD-1 expression.

In addition to PD-1, other cell surface inhibitory molecules, including lymphocyte activation gene 3 protein (LAG-3), T cell immunoglobulin domain and mucin domain-containing protein 3 (TIM-3; also known as HAVCR2) and cytotoxic T-lymphocyte associated protein 4 (CTLA-4), also play important roles in inducing T cell exhaustion and limiting the antitumor efficacy of CAR-T cell therapy. In order to evaluate whether the expression of other T cell exhaustion markers is regulated by CAR stimulation, we measured the expression of LAG-3 and TIM-3 on CAR-engineered T cells. Similar to PD-1, we found that the expression of LAG-3 and TIM-3 was significantly upregulated on both CAR19 and CAR19.αPD1 T cells following antigen stimulation, compared with nontransduced T cells. In comparison to CAR19 T cells, CAR19.αPD1 T cells expressed slightly lower LAG-3 and TIM-3 after stimulation with H292-CD19 cells. Moreover, upon SKOV3-CD19 stimulation, CAR19.αPD1 T cells had significantly lower LAG-3 expression than CAR19 T cells, whereas they had similar TIM-3 expression (FIGS. 3C, 3D, 12A, 13A-13C). In comparison, without antigen-specific stimulation, LAG-3 in CAR19 and CAR19.αPD1 T cells was expressed at a similar level and remained stable over the course of T cell expansion (FIGS. 13D and 13E).

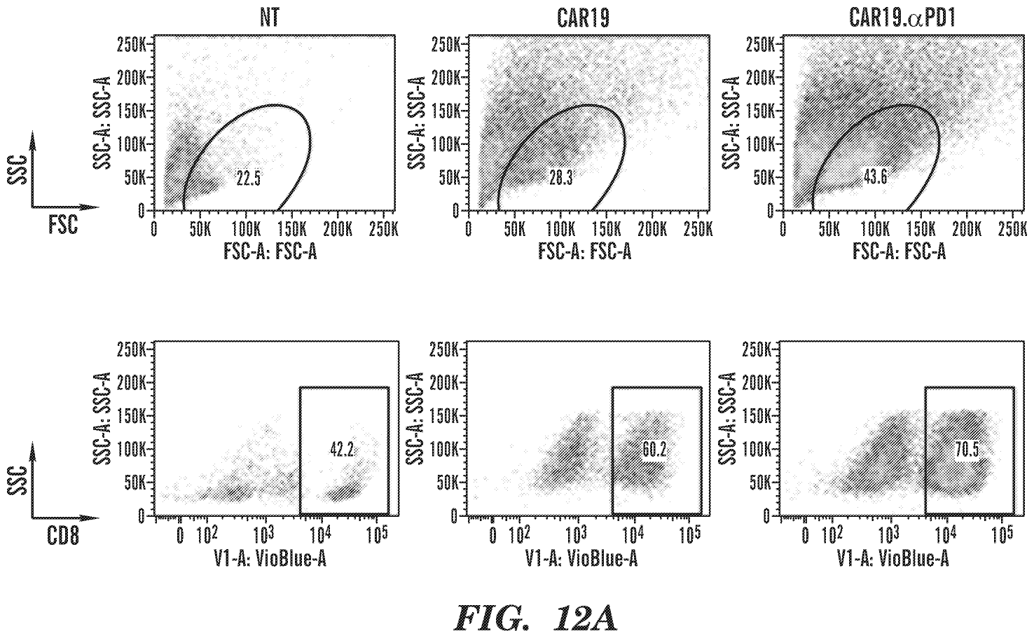

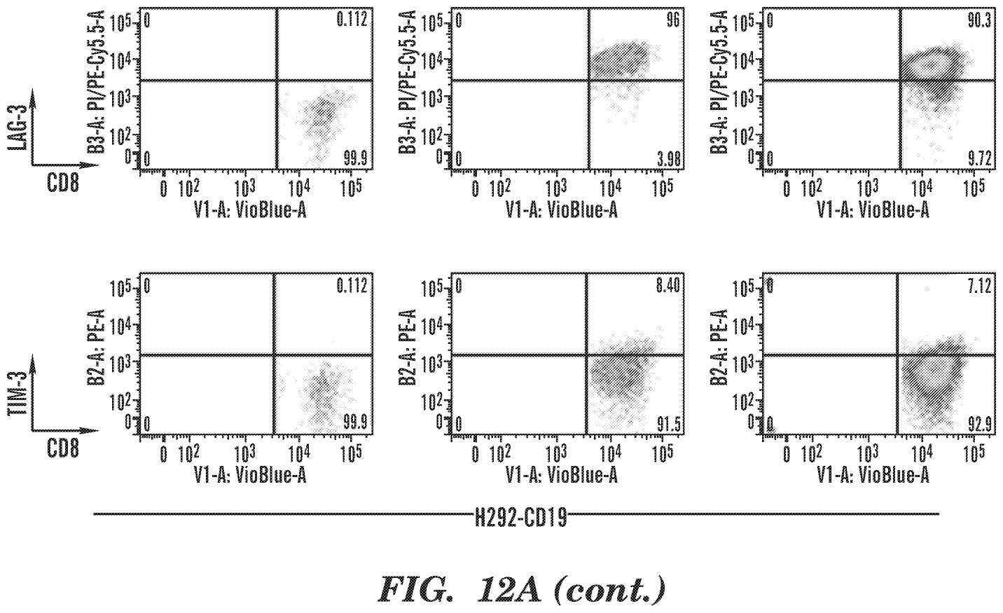

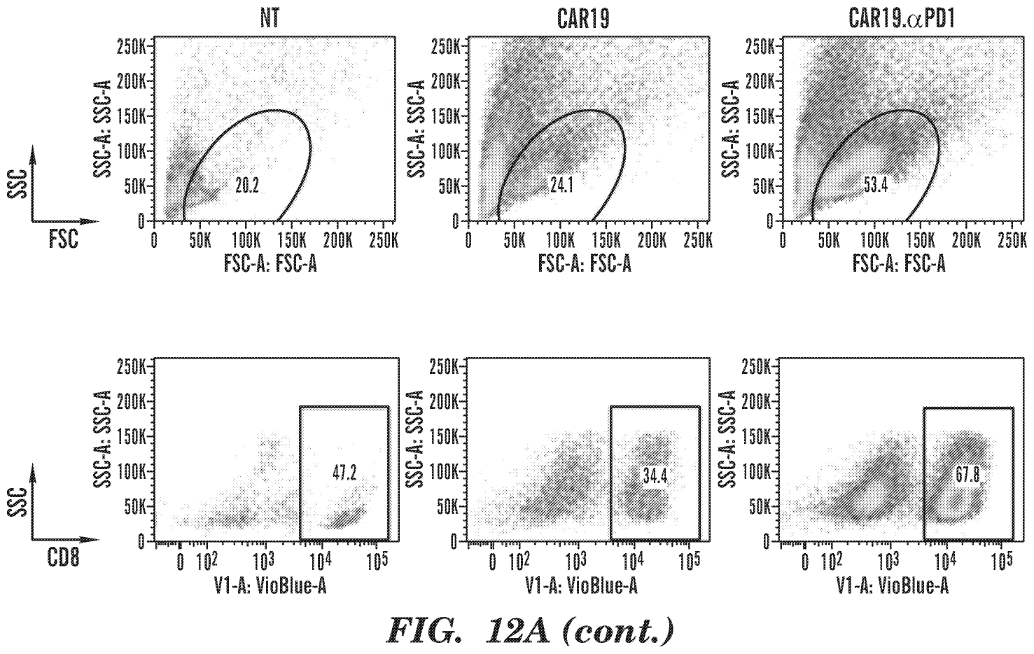

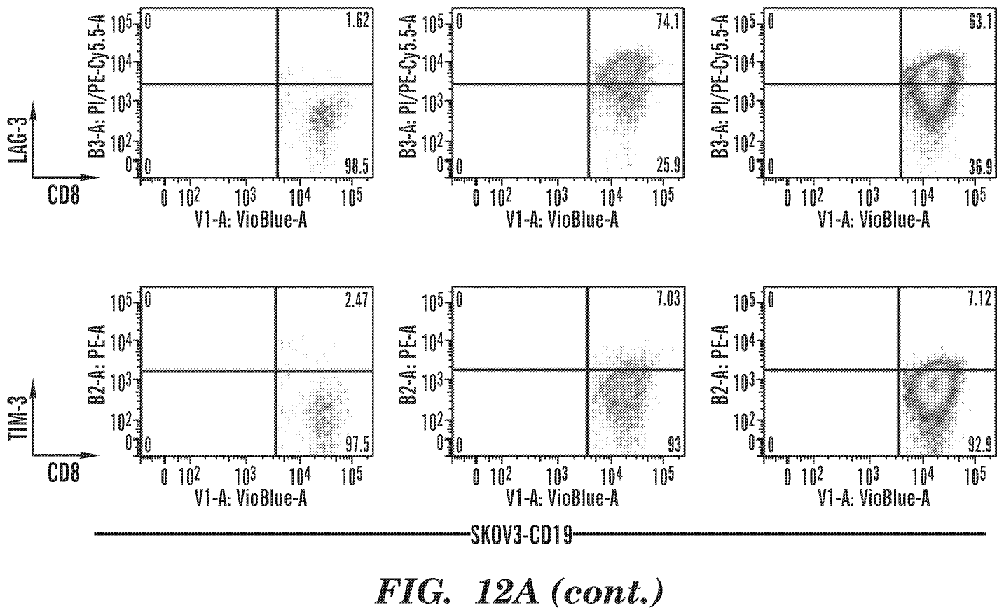

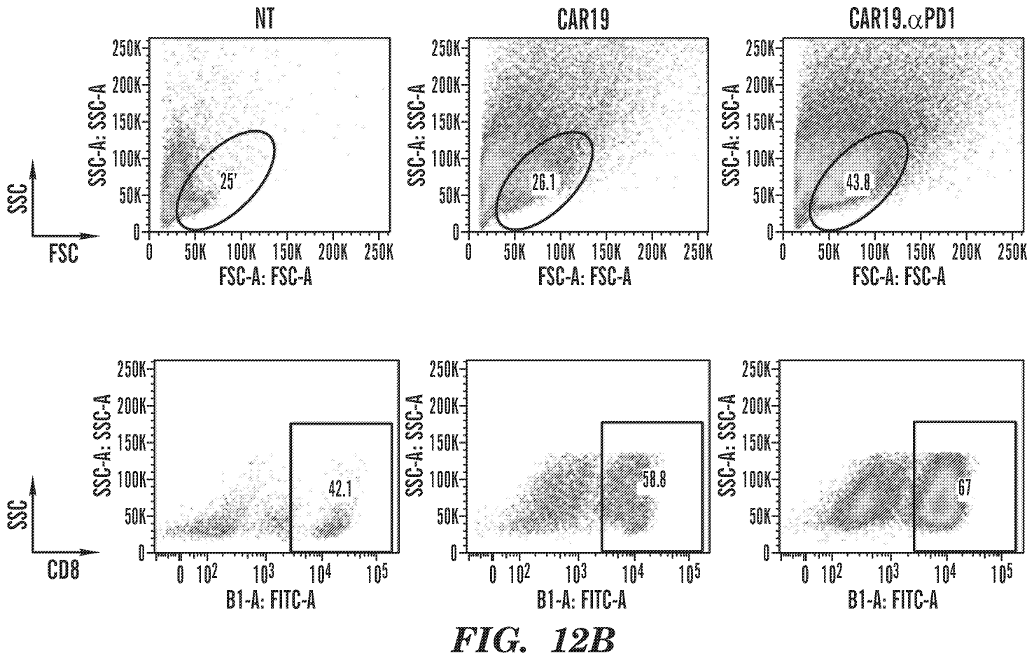

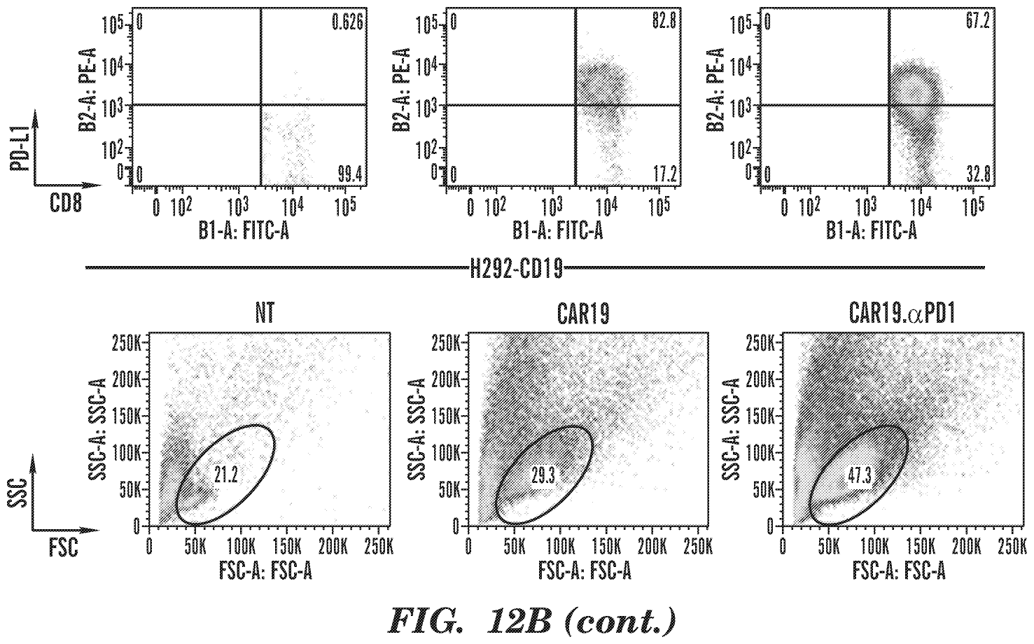

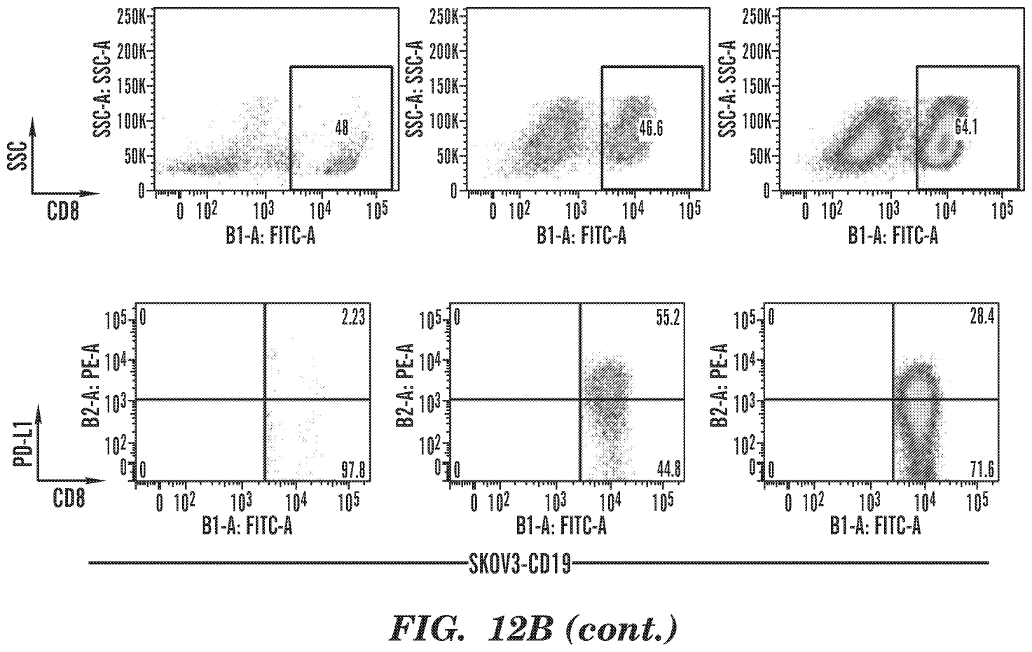

It has been shown that PD-1 blockade could promote the survival of GD2 CAR T cells after activation with the PD-L1-negative target cells, indicating that the interaction between PD-1-expressing T cells and T cells expressing PD-1 ligands, such as PD-L1, might contribute to the suppression of T cell function (Gargett T, et al 2016. GD2-specific CAR T Cells Undergo Potent Activation and Deletion Following Antigen Encounter but can be Protected From Activation-induced Cell Death by PD-1 Blockade. Molecular Therapy 24: 1135-49). Thus, in this experiment, we also measured the expression of PD-L1 in both CAR19 and CAR19.αPD1 T cells and found that it was significantly increased following antigen-specific stimulation. However, the expression of PD-L1 in CAR19.αPD1 T cells was significantly lower than that in CAR19 T cells (FIGS. 3E, 3F, and 12B).

### Anti-PD-1 Engineered CAR T Cells Exhibit Enhanced Antitumor Reactivity

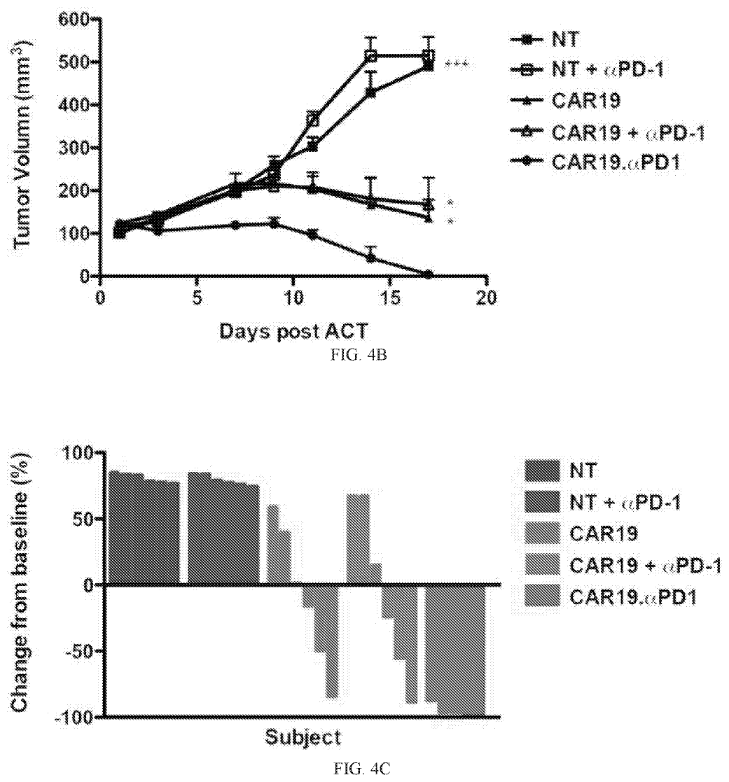

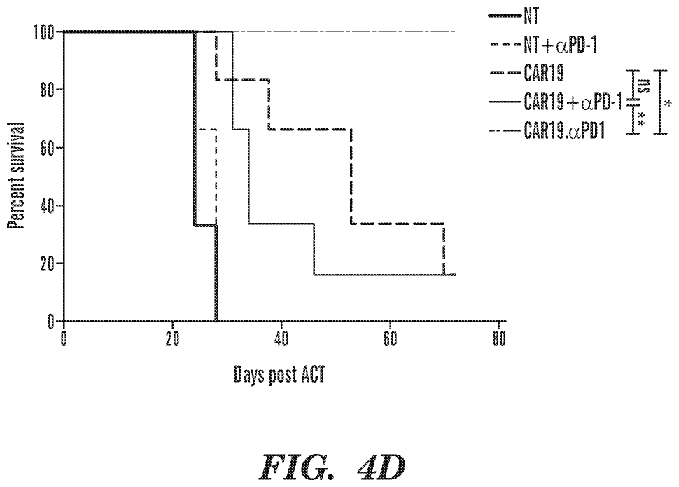

To evaluate the antitumor efficacy of CAR19.αPD1 T cells, we adoptively transferred 1×10⁶ CAR-engineered T cells into NSG mice bearing established H292-CD19 subcutaneous tumors (˜100 mm³). The experimental procedure for animal study is shown in FIG. 4A. The data in FIG. 4B demonstrate that all three anti-CD19 CAR T cell groups showed decreased tumor sizes compared to nontransduced T cells or nontransduced T cells combined with anti-PD-1 antibody treatment over the course of the experiment. However, in comparison to parental CAR19 T cells or CAR19 T cells combined with anti-PD-1 antibody treatment, CAR19.αPD1 T cell treatment significantly enhanced the antitumor effect, which became evident as early as one week after T cell infusion (FIG. 4B). Notably, 17 days after adoptive cell transfer, we observed that the tumors from mice treated with CAR19.αPD1 T cells almost disappeared. In the parental CAR19 T cell group or combination group, 4 out of 6 mice (˜70%) still had either progressive or stable disease states and only experienced a decrease in tumor size of less than 30% (FIG. 4C). The overall survival of the tumor-bearing mice was also evaluated. It showed that CAR19.αPD1 T cell treatment significantly prolonged long-term survival (100%), compared to either the parental CAR19 T cell treatment alone (17%) or the combined anti-PD-1 antibody and CAR19 T cell treatment (17%) (FIG. 4D).

Anti-PD-1 Engineered CAR T Cells can Expand More In Vivo than Parental CAR T Cells

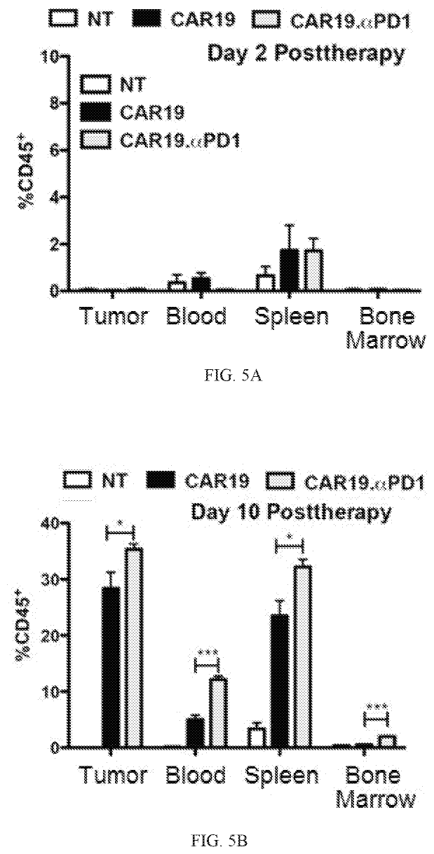

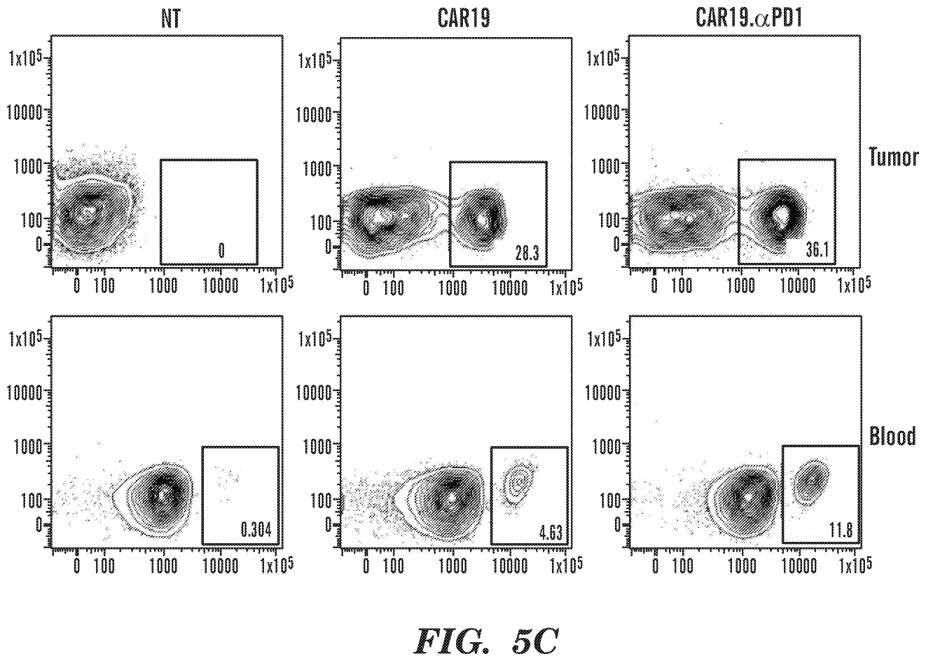

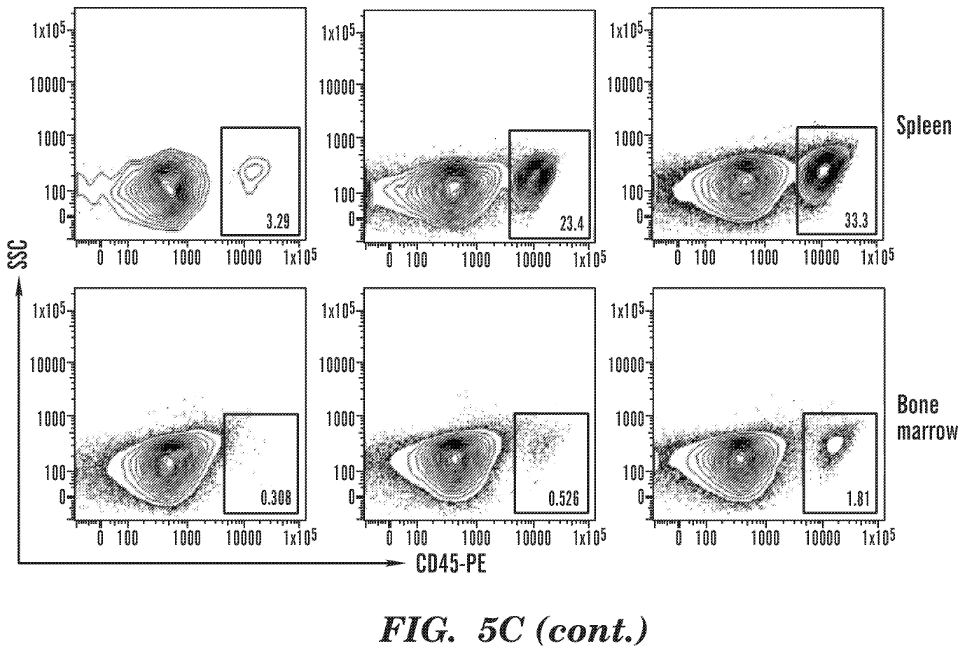

Next, the engraftment and expansion of CART cells were assessed in vivo. Two days following T cell infusion, mice were euthanized, and different organs and tissues, including the tumor, blood, spleen and bone marrow, were harvested for human T cell staining. We found that T cells in all groups had barely expanded and that less than 2% of T cells could be observed in all examined tissues. Most T cells (1-2%) homed to the spleen, while a certain percentage of T cells (0.1-0.5%) circulated were in the blood. The infiltration level of transferred T cells was low in tumor and bone marrow. In addition, the T cell percentage between the nontransduced and CAR-transduced T cells showed little difference across all examined tissues (FIG. 5A). However, one week post-T cell infusion, on day 10, we observed a significant expansion of CAR T cells in all examined tissues, whereas nontransduced T cells were barely present. Notably, consistent with our in vitro data, CAR.19.αPD1 T cells had a significantly higher expansion rate compared to parental CAR19 T cells, especially in tumor, spleen and blood (FIG. 5B and FIG. 5C).

### Anti-PD-1 Engineered CAR T Cells Lead to Reversal of T Cell Exhaustion and Higher T Cell Effector Function at the Established Tumor Site

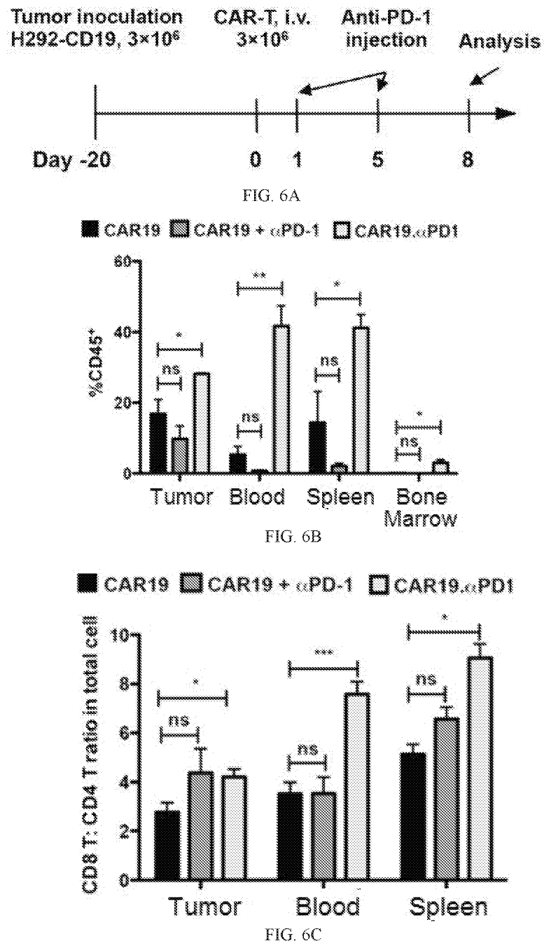

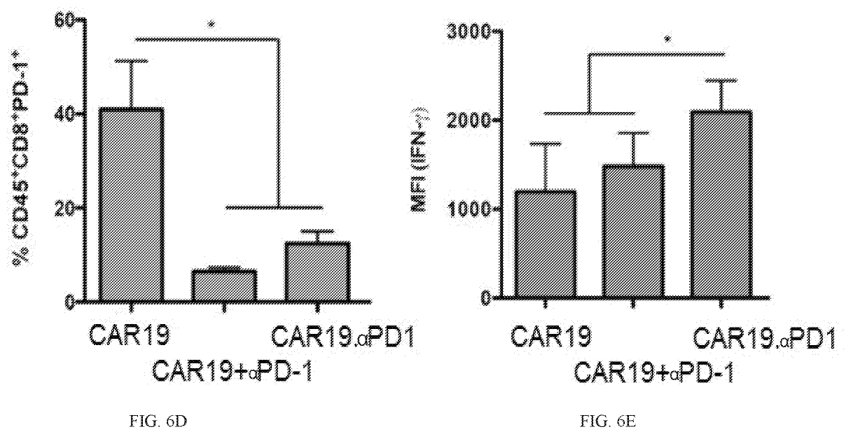

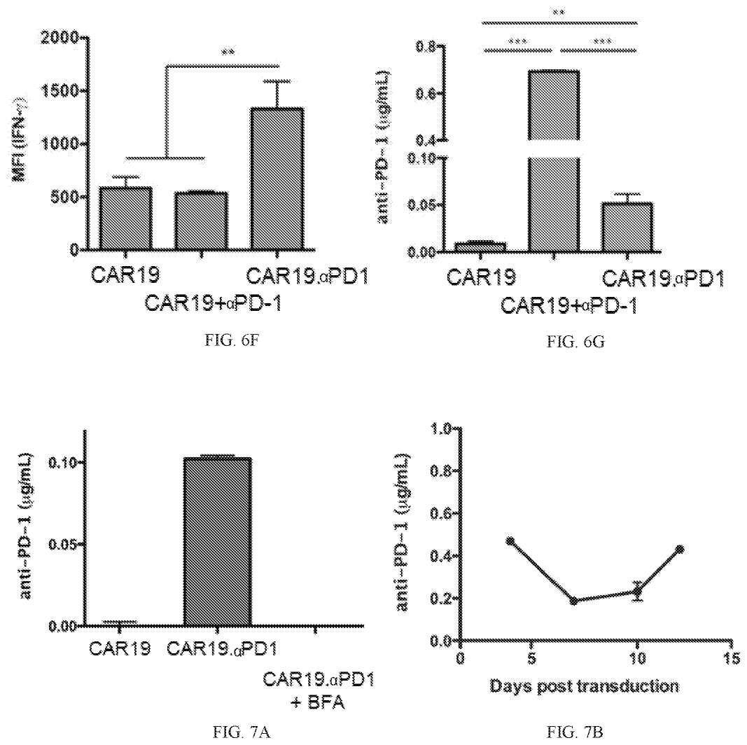

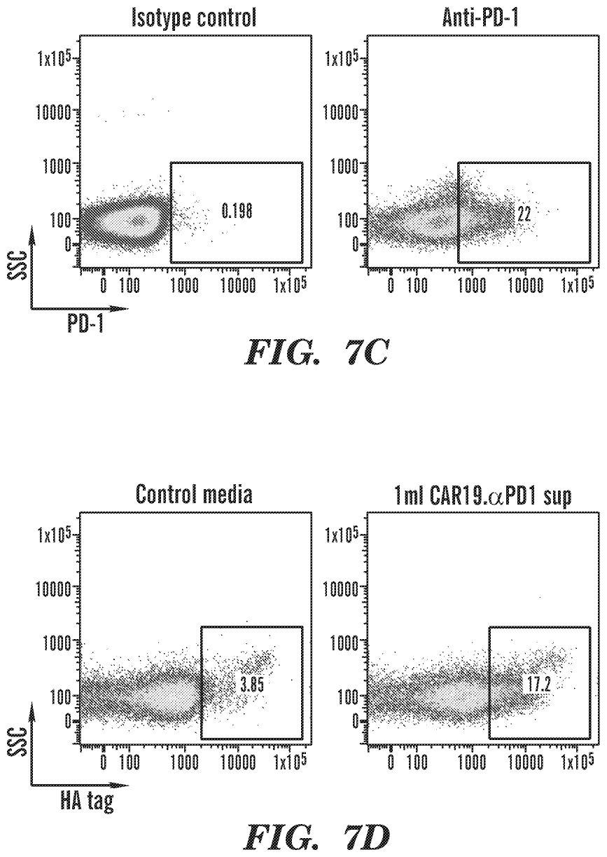

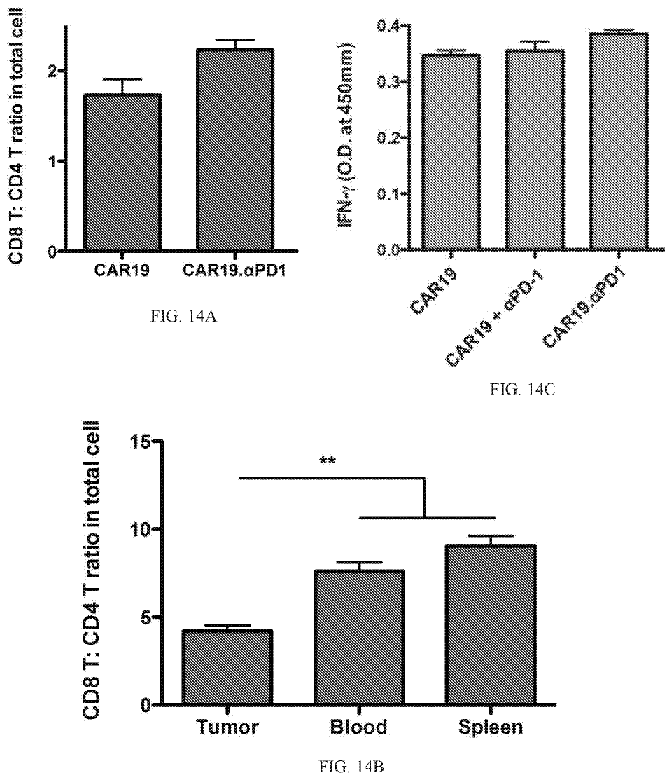

To further determine if the enhanced antitumor effects observed following CAR19.αPD1 T cell therapy are correlated with increased function of CAR T cells at the tumor site, mice were challenged with H292-CD19 tumors before receiving 3×10⁶ CAR T cells. The experimental design is shown in FIG. 6A. Eight days after T cell infusion, we euthanized the mice and analyzed T cells in tumor, blood, spleen and bone marrow, using flow cytometry. Compared to the CAR cell treatment, we observed that the injected anti-PD-1 antibody had little effect on enhancing the expansion of T cells in vivo. However, consistent with our previous observation (FIG. 5B), T cells from mice treated with the CAR19.αPD1 regimen expanded at a higher rate in tumor, blood, and spleen (FIG. 6B). It has been shown that the population of cytotoxic CD8⁺ T cells among tumor-infiltrating lymphocytes (TILs) is critical in eliciting antitumor immunity and spontaneous tumor control. Therefore, the ratio of CD8⁺ versus CD4⁺ T cells was analyzed among TILs. Compared to the parental CAR19 T cells, results showed that the CAR19.αPD1 T cells had a significantly higher ratio of CD8⁺ versus CD4⁺ T cells, whereas the combined therapy had a similar CD8⁺ versus CD4⁺ T cell ratio compared to CAR T cell monotherapy (FIG. 6C). Similarly, in the blood and spleen, the ratio of CD8⁺ versus CD4⁺ in CAR19.αPD1 T cell treatment was also significantly higher than that in parental CAR19 T cell monotherapy and combination treatment groups (FIG. 6C), though there was little difference between the CD8⁺ versus CD4⁺ T cell ratio between CAR19 and CAR19.αPD1 T cells before T cell infusion (FIG. 14A). Further, we assessed PD-1 expression on tumor-infiltrating CD8⁺ T cells and found that both the injected and secreted anti-PD-1 antibodies could significantly decrease the expression of PD-1 (FIG. 6D). We also performed the ex vivo culture and activated TILs with either anti-CD3/CD28 antibodies or target cell H292-CD19. We observed significantly higher expression of IFN-γ in adoptively transferred CAR19.αPD1 T cells, compared to either parental CAR19 T cells or CAR19 T cells combined with systemic anti-PD-1 antibody treatment. Little difference was observed in IFN-γ expression between CAR T cell monotherapy and combined therapy (FIG. 6E and FIG. 6F). Additionally, we measured the expression of IFN-γ and anti-PD-1 antibodies in the sera and found little difference in IFN-γ expression among all groups (FIG. 14C). Notably, compared to CAR19 T cell treatment, CAR19.αPD1 T cell therapy had significantly higher anti-PD-1 concentration in the sera, although the concentration was more than 15-fold lower than that with systemic anti-PD-1 antibody injection (FIG. 6G).

Adoptive T cell therapy has become a promising method of immunotherapy. It has achieved successful responses in patients with hematopoietic malignancies. However, the outcome has been less promising in the treatment of solid tumors, partly owing to the immunosuppressive properties and establishment of an immunosuppressive microenvironment. The PD-1/PD-L1 regulatory pathway has demonstrated particularly antagonistic effects on the antitumor response of TILs. Solid tumors with poor prognosis showed upregulation of PD-L1 expression, while TILs were shown to have PD-1 upregulation. The combined effect of these two results in tumor escape. However, this can be disrupted by the use of checkpoint inhibitors (CPIs) targeting the PD-1/PD-L1 pathway. As a result, the ensuing research was designed to investigate the effects of PD-1/PD-L1 blockade in infused CAR T cells, which showed upregulation of PD-1 after activation.

Despite other methods of PD-1/PD-L1 inhibition, such as cell intrinsic PD-1 shRNA and PD-1 dominant negative receptor, treatment with PD-1 or PD-L1 antibody has long been a topic of interest and extensively studied in both animal models and clinical trials. Indeed, both antibodies have resulted in a marked inhibition of tumor growth. However, antibody treatment has multiple limitations. For example, it requires multiple and continuous antibody administration to obtain a sustained efficacy. Also, the large size of antibodies prevents them from entering the tumor mass and encountering the infiltrated PD-1-positive T cells. To account for these inefficiencies, multiple high-dose treatments with immunomodulatory drugs or antibodies are required, but this can result in side effects that range from mild diarrhea to autoimmune hepatitis, pneumonitis and colitis. Moreover, it has been shown that the Fc portion of antibodies may cause immune cell depletion by activating cytotoxic signals within macrophages and natural killer cells, which usually express FcαRI and FcγRIIIA/FcγRIIC, respectively. Therefore, in this study, we focused our efforts on engineering CAR T cells to secrete and deliver high concentrations of human scFvs against PD-1, aiming to change the immunosuppressive tumor microenvironment, prevent tumor-induced hypofunctionality and enhance the antitumor immunity of infused CAR T cells.

Herein, we engineered human anti-CD19 CAR T cells that secrete human anti-PD-1 scFvs and demonstrated that anti-PD-1 scFv could be efficiently expressed and secreted by CAR19.αPD1 T cells. The secreted scFvs successfully bound to PD-1 on the cell surface and reversed the inhibitory effects of PD-1/PD-L1 interaction on T cell function. PD-1 blockade by constitutively secreted anti-PD-1 scFv decreased T cell exhaustion and significantly enhanced T cell proliferation and effector function in vitro. Our study using xenograft mouse models also demonstrated that CAR19.αPD1 T cells, when compared to parental CAR19 T cells, further enhanced antitumor activity and prolonged overall survival. Mechanistically, we observed that CAR19.αPD1 T cells had greater in vivo expansion. In addition, at the local tumor site, CAR19.αPD1 T cells were shown to be less exhausted and more functional than parental CAR19 T cells.

The engagement of PD-1 and its ligand PD-L1 or PD-L2 transduces an inhibitory signal and suppresses T cell function in the presence of TCR or BCR activation. In this study, the presence of recombinant human PD-L1 protein (rhPD-L1) significantly inhibited T cell activation in an in vitro activation assay. To examine the binding and blocking activity of anti-PD-1 say secreted by CAR19.αPD1 cells, we cultured the T cells with cell culture supernatant from either CAR19 T cells or CAR19.αPD-1 T cells in the presence of rhPD-L1 protein. We observed that the supernatant from CAR19.αPD1 T cells rescued T cell function and significantly increased IFN-γ production, indicating that secreted anti-PD-1 could successfully bind to PD-1 and reverse the inhibitory effects of the PD-1/PD-L1 interaction on T cell function.

The PD-1/PD-L1 pathway involves the regulation of cytokine production by T cells, inhibiting production of IFN-γ, TNF-α and IL-2. PD-1 expression of human GD2 and anti-HER2 CAR T cells has been shown to increase following antigen-specific activation, and PD-1 blockade has been shown to enhance T cell effector function and increase the production of IFN-γ in the presence of PD-L1⁺ target cells. Therefore, in this study, to compare the functional capacity of CAR19 T and CAR19.αPD1 T cells, we cocultured T cells with a PD-L1⁺ cancer cell line, H292-CD19 or SKOV3-CD19, and found that the anti-PD-1-secreting CAR19 T cells produced a significantly higher level of than parental CAR19 T cells. In addition to cytokine production, PD-1 can also inhibit T cell proliferation. With CAR-specific stimulation in the presence of PD-L1⁺ cancer cells, we found that CAR19.αPD1 T cells had a significantly higher proliferation rate than the parental CAR19 T cells. Taken together, these data imply that PD-1/PD-L1 signaling blockade results in more functional CAR19.αPD1 T cells with higher proliferation capacity compared to CAR19 T cells alone.

To better understand how secreted anti-PD-1 affects the function of CAR19.αPD1 T cells, we exposed CAR19 T cells and CAR19.αPD1 T cells to PD-L1⁺ target cells and examined the expression of T cell exhaustion markers, including PD-1, LAG-3 and TIM-3. We observed significantly lower PD-1 expression on CAR19.αPD1 T cells, as well as lower expression of other exhaustion markers, such as LAG-3, compared with parental CAR19 T cells. The decreased expression of PD-1 in CAR19.αPD1 T cells may be caused by the dual effects of antibody blockade and downregulation of PD-1 surface expression. PD-1 upregulation on tumor-infiltrating T cells was reported to be a major contributor to T cell exhaustion in high PD-L1-expressing tumors. Downregulation of PD-1 may contribute to reversion of T cell exhaustion and enhanced T cell effector function, which is supported by increased IFN-γ production of CAR19.αPD1 T cells. In addition, the lower expression level of other exhaustion makers, such as LAG-3, may also contribute to the higher function of CAR19.αPD1 T cells upon antigen stimulation. Our observation is consistent with a recent study, demonstrating that co-expression of multiple inhibitory receptors is a cardinal feature of T cell exhaustion. Moreover, we found that PD-L1 expression was significantly increased on CAR T cells with antigen-specific stimulation, which may also contribute to T cell exhaustion through T cell-T cell interaction. Notably, in comparison, we observed that the expression level of PD-L1 on CAR19.αPD1 T cells was significantly lower. These data suggest that the inhibited upregulation of PD-1 and PD-L1 expression on CAR19.αPD1 T cells may contribute to the reduction of tumor cell-induced and/or T cell-induced exhaustion, thereby further enhancing T cell effector function and its antitumor immunity.

Our in vivo study showed that the tumor growth could be inhibited by CAR T cell treatment, irrespective of PD-1/PD-L1 blockade. Compared to CAR19 T cell treatment or combined CAR19 T cell and systemic anti-PD-1 antibody treatment, in which 67% of the mice still had either stable or progressive disease, we observed that CAR19.αPD1 T cell treatment achieved more than 90% tumor eradication in about two weeks. To understand the underlying mechanism of enhanced antitumor efficacy of CAR19.αPD1 T cells, we analyzed the expansion of adoptively transferred T cells in vivo. Consistent with our in vitro data, we found that the anti-PD-1-secreting CAR T cells were expanded significantly more than parental CAR T cells in all examined tissues, including tumor, blood, spleen and bone marrow. Moreover, the population of cytotoxic CD8⁺ T cells among TILs is critical in eliciting antitumor immunity. A previous study demonstrated that PD-1 signaling is involved in regulating the expansion and function of CD8⁺ TILs. In this study, the larger population of CD8⁺ TILs expresses IFN-γ when stimulated ex vivo and the higher ratio of CD8⁺ versus CD4⁺ TILs in the CAR19.αPD1 T cell group implies that CAR19.αPD1 T cells are more functional and expandable in vivo compared to parental CAR19 T cells.

Interestingly, in this study, we demonstrated that systemic anti-PD-1 antibody injection has little effect on enhancing the antitumor efficacy of CAR T cell therapy. In a syngeneic HER2⁺ self-antigen tumor model, recent studies have demonstrated that a high-dosage (250 μg/mouse of anti-PD-1 antibody) PD-1 blockade was capable of enhancing the antitumor activity of anti-HER2 CAR T cells in the treatment of breast cancer. However, a lower dosage (200 μg/mouse) of anti-PD-1 antibody showed a limited effect on CAR T cell therapy. In the present study, with a low-dose (125 μg/mouse) injection, the anti-PD-1 antibody failed to inhibit tumor growth or enhance the antitumor efficacy of CAR T cells. This observation indicates that a large dose of anti-PD-1 antibody, which often causes systemic toxicity, may be required to achieve substantial antitumor efficacy. We measured the amount of circulating anti-PD-1 antibodies and found a significant amount of circulating injected antibody (˜0.7 μg/ml) in the combination treatment group and a 15-fold lower amount in the CAR19.αPD1 T cell treatment group. Although both administered and self-secreting anti-PD-1 antibodies efficiently decreased and blocked the PD-1 expression in CD8⁺ T cells in vivo, systemically injected anti-PD-1 antibody had little effect on increasing the population of cytolytic CD8⁺ TILs or enhancing IFN-γ production of TILs upon ex vivo stimulation. This result suggests that the injected antibody has little effect on augmenting infused T cell function at the present dose. It also explains our observed failure of injected PD-1 blockade in enhancing the antitumor activity of CAR T cell therapy. Given the low concentration of secreted anti-PD-1 and the augmented effector function at the local tumor tissue, the anti-PD-1 secreted by CAR T cells may provide a safer and more potent approach in blocking PD-1 signaling and enhancing the functional capacity of CAR T cells.

In conclusion, CAR19.αPD1 T cells exhibited alleviated T cell exhaustion, enhanced T cell expansion, and improved CAR T cell treatment of human solid tumors in a xenograft mouse model. In an immune competent condition, we speculate that anti-PD-1-engineered CAR T cells might be more powerful in inducing tumor eradication given the durable effect of PD-1 blockade on modulating the tumor microenvironment. In addition, we foresee that engineering the anti-PD-1 scFv into CAR constructs targeting other tumor-associated antigens, such as mesothelia or HER-2 for the treatment of ovarian cancer or breast cancer, which usually have high PD-L1 expression, is among the next steps that should be explored to achieve better antitumor immunotherapy.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can he practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A cell comprising a nucleic acid encoding a chimeric antigen receptor (CAR) and a checkpoint inhibitor (CPI) or nucleic acids encoding a CAR and a CPI.

2. The cell of claim 1, wherein the CAR targets cluster of differentiation (CD) 19, CD22, CD23, myeloproliferative leukemia protein (MPL), CD30, CD32, CD20, CD70, CD79b, CD99, CD123, CD138, CD179b, CD200R, CD276, CD324, Fc receptor-like 5 (FcRH5), CD171, CS-1 (signaling lymphocytic activation molecule family 7, SLAMF7), C-type lectin-like molecule-1 (CLL-1), CD33, cadherin 1, cadherin 6, cadherin 16, cadherin 17, cadherin 19, epidermal growth factor receptor variant III (EGFRviii), ganglioside GD2, ganglioside GD3, human leukocyte antigen A2 (HLA-A2), B-cell maturation antigen (BCMA), Tn antigen, prostate-specific membrane antigen (PSMA), receptor tyrosine kinase like orphan receptor 1 (ROR1), FMS-like tyrosine kinase 3 (FLT3), fibroblast activation protein (FAP), tumor-associated glycoprotein (TAG)-72, CD38, CD44v6, carcinoembryonic antigen (CEA), epithelial cell adhesion molecule (EpCAM), KIT, interleukin-13 receptor subunit alpha-2 (IL-13Ra2), interleukin-11 receptor subunit alpha (IL11Ra), Mesothelin, prostate stem cell antigen (PSCA), vascular endothelial growth factor receptor 2 (VEGFR2), Lewis Y, CD24, platelet derived growth factor receptor beta (PDGFR-beta), Protease Serine 21 (PRSS21), sialyl glycolipid stage-specific embryonic antigen 4 (SSEA-4), Fc region of an immunoglobulin, tissue factor, folate receptor alpha, epidermal growth factor receptor 2 (ERBB2), mucin 1 (MUC1), epidermal growth factor receptor (EGFR), neural small adhesion molecule (NCAM), Prostase, prostatic acid phosphatase (PAP), elongation factor 2 mutated (ELF2M), Ephrin B2, insulin-like growth factor I receptor (IGF-I receptor), carbonic anhydrase IX (CAIX), latent membrane protein 2 (LMP2), melanocyte protein gp100, bcr-abl, tyrosinase, erythropoietin-producing hepatocellular carcinoma A2 (EphA2), fucosylated monosialoganglioside (Fucosyl GM1), sialyl Lewis a (sLea), ganglioside GM3, transglutaminase 5 (TGS5), high molecular weight melanoma-associated antigen (HMWMAA), o-acetyl-GD2 ganglioside, folate receptor beta, TEM1/CD248, tumor endothelial marker 7-related (TEM7R), claudin 6 (CLDN6), thyroid stimulating hormone receptor (TSHR), T cell receptor (TCR)-beta1 constant chain, TCR beta2 constant chain, TCR gamma-delta, G protein-coupled receptor class C group 5 member D (GPRC5D), CXORF61 protein, CD97, CD179a, anaplastic lymphoma kinase (ALK), Polysialic acid, placenta specific 1 (PLAC1), carbohydrate antigen GloboH, breast differentiation antigen NY-BR-1, uroplakin-2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 family member K (LY6K), olfactory receptor family 51 subfamily E member 2 (OR51E2), T-cell receptor γ-chain alternate reading-frame protein (TARP), Wilms tumor antigen 1 protein (WT1), cancer-testis antigen NY-ESO-1, cancer-testis antigen LAGE-1a, legumain, human papillomavirus (HPV) E6, HPV E7, Human T-lymphotrophic viruses (HTLV1)-Tax, Kaposi's sarcoma-associated herpesvirus glycoprotein (KSHV) K8.1 protein, Epstein-Barr virus (EBV)-encoded glycoprotein 350 (EBB gp350), HIV1-envelop glycoprotein gp120, multiplex automated genome engineering (MAGE)-A1, translocation-Ets-leukemia virus (ETV) protein 6-AML, sperm protein 17, X Antigen Family Member (XAGE)1, transmembrane tyrosine-protein kinase receptor Tie 2, melanoma cancer-testis antigen MAD-CT-1, melanoma cancer-testis antigen MAD-CT-2, Fos-related antigen 1, p53, p53 mutant, prostein, survivin and telomerase, prostate cancer tumour antigen-1 (PCTA-1)/Galectin 8, MelanA/MART1, Ras mutant, human telomerase reverse transcriptase (hTERT), delta-like 3 (DLL3), Trophoblast cell surface antigen 2 (TROP2), protein tyrosine kinase-7 (PTK7), Guanylyl Cyclase C (GCC), alpha-fetoprotein (AFP), sarcoma translocation breakpoints, melanoma inhibitor of apoptosis (ML-IAP), ERG (TMPRSS2 ETS fusion gene), N-acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), tyrosinase-related protein 2 (TRP-2), Cytochrome P4501B1 (CYP1B1), CCCTC-Binding Factor (Zinc Finger Protein)-Like (BORIS or Brother of the Regulator of Imprinted Sites), squamous Cell Carcinoma Antigen Recognized By T Cells 3 (SART3), PAX5, proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), RU2, intestinal carboxyl esterase, heat shock protein 70-2 mutated (mut hsp70-2), CD79a, CD72, leukocyte-associated immunoglobulin-like receptor 1 (LAIR1), Fc fragment of IgA receptor (FCAR), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), immunoglobulin lambda-like polypeptide 1 (IGLL1), FITC, Leutenizing hormone receptor (LHR), Follicle stimulating hormone receptor (FSHR), Chorionic Gonadotropin Hormone receptor (CGHR), CC chemokine receptor 4 (CCR4), signaling lymphocyte activation molecule (SLAM) family member 6 (SLAMF6), SLAMF4, or combinations thereof.

3. The cell of claim 1, wherein the checkpoint inhibitor targets programmed cell death protein 1 (PD-1).

4. The cell of claim 3, wherein the checkpoint inhibitor is an anti-PD-1 scFv.

5. The cell of claim 1, wherein the checkpoint inhibitor targets any one or more of PD-1, lymphocyte-activation gene 3 (LAG-3), T-cell immunoglobulin and mucin domain-3 (TIM3), B7-H1, CD160, P1H, 2B4, carcinoembryonic antigen related cell adhesion molecule 1 (CEACAM-1), CEACAM-3, CEACAM-5, T cell immunoreceptor with Ig and ITIM domains (TIGIT), cytotoxic T-lymphocyte-associated protein 4 (CTLA-4), B- and T-lymphocyte attenuator (BTLA), and LAIR1.

6. The cell of claim 1, wherein the cell is a T-lymphocyte cell (T-cell).

7. The cell of claim 1, wherein the cell is a Natural Killer (NK) cell.

8. The cell of claim 1, wherein the CPI is constitutively expressed.

9. The cell of claim 4, wherein the anti-PD-1 scFv is constitutively expressed.

10. A nucleic acid comprising a first polynucleotide encoding a chimeric antigen receptor (CAR) and a second polynucleotide encoding a checkpoint inhibitor (CPI).

11. Polypeptides encoded by the nucleic acid of claim 10.

12. A vector comprising the nucleic acid of claim 10.

13. A pharmaceutical composition, comprising the cell of claim 1.

14. A method for treating cancer comprising administering to a subject in need thereof, a therapeutically effective amount of the cell of claim 1.

15. The method of claim 14, wherein the cancer is lung cancer.

16. The method of claim 14, further comprising administering to the subject a therapeutically effective amount of an existing therapy comprising chemotherapy or radiation.

17. The method of claim 16, wherein the cell and the existing therapy are administered sequentially or simultaneously.


SEQUENCE LISTING (1 sequence)

SEQ ID NO: 1 - protein, 8 aa, Artificial Sequence
  synthetic construct
  MISC_FEATURE (2)..(2): Xaa can be Val or Ile
  misc_feature (4)..(4): Xaa can be any naturally occurring amino acid
DXEXNPGP